(19) United States
(12) Patent Application Publication
(10) Pub. No.: US 2026/0146025 A1
(43) Pub. Date: May 28, 2026

(54) CRYSTALLINE POLYMORPHS OF MITAPIVAT AND MITAPIVAT HEMISULFATE

(71) Applicant: TIANISH LABORATORIES PRIVATE LIMITED, Hyderabad (IN)

(72) Inventors: Ramakoteswara Rao Jetti, Hyderabad (IN); Shaunak Chakraborty, Hyderabad (IN); Narasimha Murty Pilli, Hyderabad (IN); Dhaneswara Venkata Jesunadh Karri, Hyderabad (IN); Nitin Shimpi, Hyderabad (IN); Sureshbabu Jayachandra, Hyderabad (IN); B.A. Ramireddy, Hyderabad (IN); Ramamohana Rao Golivi, Hyderabad (IN); Subramanyam Dandala, Hyderabad (IN); Chandra has Khanduri, Hyderabad (IN); Ramesh Batharaju, Hyderabad (IN)

(73) Assignee: TIANISH LABORATORIES PRIVATE LIMITED, Hyderabad (IN)

(21) Appl. No.: 19/121,759

(22) PCT Filed: Oct. 16, 2023
(86) PCT No.: PCT/IN2023/050948

§ 371 Date: Apr. 16, 2025

(30) Foreign Application Priority Data
Oct. 17, 2022 (IN) 202241059196
Oct. 22, 2022 (IN) 202241060563
Dec. 1, 2022 (IN) 202241069419
Feb. 14, 2023 (IN) 202341009761

(51) Int. Cl. C07D 215/36

(52) U.S. Cl. CPC C07D 215/36

(57) ABSTRACT

The present application describes crystalline forms of mitapivat, amorphous mitapivat mono sulfate, crystalline forms of mitapivat mono sulfate, and crystalline forms of mitapivat hemi sulfate. The present application also describes a process for the preparation of crystalline forms of mitapivat, mitapivat hemi sulfate, mitapivat mono sulfate and amorphous mitapivat mono sulfate.

### CROSS REFERENCE TO THE RELATED APPLICATIONS

This application claims the benefit of the earlier filing date of Indian Provisional Patent Application No. 202241059196 filed on Oct. 17, 2022, Indian Provisional Patent Application No. 202241060563 filed on Oct. 22, 2022, Indian Provisional Patent Application No. 202241069419 filed on Dec. 1, 2022, and Indian Provisional Patent Application No. 202341009761 filed on Feb. 14, 2023; the entire contents of each of which are incorporated by reference herein.

### BACKGROUND OF THE INVENTION

### Field of the Disclosure

The present disclosure relates to novel crystalline forms of mitapivat and mitapivat salts. The present disclosure also relates to a process for the preparation of mitapivat and its salt.

### Description of the Related Art

Mitapivat is a Pyruvate kinase R (PKR) activator and is developed by Agios for the treatment of inborn errors of metabolism (IEMs) like Pyruvate kinase deficiencies, thalassemia and sickle cell disease. Mitapivat is marketed under the brand name PYRUKYND in the U.S. The active ingredient of PYRUKYND is mitapivat, present as mitapivat sulfate. The chemical name of mitapivat sulfate is 8-quinolinesulfonamide, N-[4-[[4(cyclopropylmethyl)-1-piperazinyl]carbonyl]phenyl]-, sulfate and molecular formula is (C₂₄H₂₆N₄SO₃)₂·H₂SO₄. The chemical structure of mitapivat sulfate is:

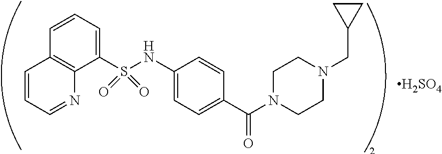

Mitapivat first disclosed in International Publication No. WO2011/002817. International Publication No. WO2019/104134 describes amorphous and different crystalline forms of Mitapivat hemi sulfate of formula.

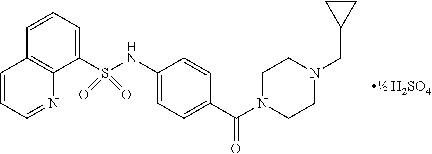

Different polymorphs may provide different advantages in a variety of capacities, for example, in ease of formulation, stability of the polymorphic form, stability of the formulation, and in pharmacokinetic profiles. These advantages may arise from the different properties present in each polymorph. The present invention provides novel polymorphic forms of mitapivat, mitapivat hemi sulfate, mitapivat mono sulfate and process for the preparation thereof.

### SUMMARY OF THE DISCLOSURE

A first aspect of the present invention is to provide crystalline mitapivat hemi sulfate Form I.

Other aspect of the present invention is to provide a process for the preparation of crystalline mitapivat hemi sulfate Form I, comprising the steps of:
a) providing mitapivat in a mixture of solvents and adding sulfuric acid;
b) heating the reaction mixture;
c) optionally reducing the volume of the reaction mass by removing the solvent;
d) adding organic solvent to the reaction mixture; and
e) isolating crystalline mitapivat hemi sulfate Form I.

A second aspect of the present invention is to provide crystalline mitapivat hemi sulfate Form II.

Other aspect of the present invention is to provide a process for the preparation of crystalline mitapivat hemi sulfate Form II comprising the steps of:
a) providing mitapivat in a mixture of solvents and adding sulfuric acid;
b) heating the reaction mixture;
c) optionally reducing the volume of the reaction mass by removing the solvent;
d) cooling the reaction mixture obtained in step (c); and
e) isolating crystalline mitapivat hemi sulfate Form II.

A third aspect of the present invention is to provide crystalline mitapivat hemi sulfate Form III.

Other aspect of the present invention is to provide a process for the preparation of crystalline mitapivat hemi sulfate Form III comprising the steps of:
a) providing mitapivat in a mixture of solvents and adding sulfuric acid;
b) heating the reaction mixture obtained in step (a);
c) optionally reducing the reaction mass by removing the solvent; and
d) cooling the reaction mixture obtained in step (c);
e) isolating and drying to get crystalline mitapivat hemi sulfate Form III.

Another aspect of the present disclosure provides a process for the preparation of crystalline mitapivat hemi sulfate Form III by drying crystalline mitapivat hemi sulfate Form II.

A fourth aspect of the present invention is to provide crystalline mitapivat hemi sulfate Form IV.

Another aspect of the present invention is to provide a process for the preparation of crystalline mitapivat hemi sulfate Form IV comprising the steps of:
a) dissolving mitapivat in acetic acid;
b) adding sulfuric acid diluted with acetic acid;
c) adding an organic solvent to the reaction mixture obtained in step (b); and
d) isolating crystalline mitapivat hemi sulfate Form IV.

Other aspect of the present invention is to provide a process for the preparation of crystalline mitapivat hemi sulfate Form IV comprising the steps of:
a) dissolving mitapivat hemi sulfate in acetic acid;
b) adding an organic solvent; and
c) isolating crystalline mitapivat hemi sulfate Form IV.

A fifth aspect of the present invention is to provide crystalline mitapivat hemi sulfate Form V.

Another aspect of the present invention is to provide a process for the preparation of crystalline mitapivat hemi sulfate Form V comprising the steps of:
a) suspending crystalline mitapivat hemi sulfate Form IV in a polar solvent; and
b) isolating crystalline mitapivat hemi sulfate Form V.

A sixth aspect of the present invention is to provide crystalline mitapivat hemi sulfate Form VI.

Another aspect of the present invention is to provide a process for the preparation of crystalline mitapivat hemi sulfate Form VI comprising the steps of:
a) dissolving mitapivat hemi sulfate in a halogenated alcohol solvent;
b) adding water to the above reaction mixture; and
c) isolating crystalline mitapivat hemi sulfate Form VI.

Other aspect of the present invention is to provide a process for the preparation of crystalline mitapivat hemi sulfate Form VI comprising the steps of:
a) diluting sulfuric acid with water and adding the seeds of crystalline mitapivat hemi sulfate Form VI to the diluted sulfuric acid solution;
b) dissolving mitapivat in a halogenated alcohol solvent and adding to the reaction mixture obtained in step (a) or vice-versa; and
c) isolating crystalline mitapivat hemi sulfate Form VI.

A seventh aspect of the present invention is to provide crystalline mitapivat hemi sulfate Form VII.

Other aspect of the present invention is to provide a process for the preparation of crystalline mitapivat hemi sulfate Form VII comprising the steps of:
a) dissolving mitapivat in a halogenated alcohol solvent;
b) adding water to the reaction mixture obtained in step (a) and cooling;
c) optionally adding seeds of crystalline mitapivat hemi sulfate VII; and
d) adding sulfuric acid;
e) isolating crystalline mitapivat hemi sulfate Form VII.

In other aspect, the present invention provides a process for the preparation of crystalline mitapivat hemi sulfate Form VII comprising the steps of:
a) dissolving mitapivat hemi sulfate in a halogenated alcohol solvent;
b) reducing the volume of the reaction mixture obtained in step (a);
c) adding water to reaction mixture obtained in step (b);
d) subjecting the reaction mixture obtained in step (c) to sonication; and
e) isolating the crystalline mitapivat hemi sulfate Form VII.

Another aspect of the present invention is to provide a process for the preparation of crystalline mitapivat hemi sulfate Form VII comprising the steps of:
a) adding seeds of crystalline mitapivat hemi sulfate Form VII to water;
b) dissolving mitapivat hemi sulfate in a halogenated alcohol solvent and adding to the reaction mixture obtained in step (a) or vice-versa; and
c) isolating crystalline mitapivat hemi sulfate Form VII.

In other aspect, the present invention provides a process for the preparation of crystalline mitapivat hemi sulfate Form VII comprising the steps of:
a) diluting sulfuric acid with water;
b) dissolving mitapivat in a halogenated alcohol solvent and adding to the sulfuric acid solution obtained in step (a) or vice-versa;
c) adding water to the reaction mixture obtained in step (b); and
d) isolating crystalline mitapivat hemi sulfate Form VII.

Another aspect of the present invention is to provide a process for the preparation of crystalline mitapivat hemi sulfate Form VII comprising the steps of:
a) dissolving mitapivat in a halogenated alcohol solvent;
b) adding water to the reaction mixture obtained in step (a) and adding sulfuric acid diluted with water;
c) adding the seeds of crystalline mitapivat hemi sulfate VII; and
d) isolating crystalline mitapivat hemi sulfate Form VII.

An eighth aspect of the present invention is to provide crystalline mitapivat hemi sulfate Form VIII.

Other aspect of the present invention is to provide a process for the preparation of crystalline mitapivat hemi sulfate Form VIII comprising drying crystalline mitapivat hemi sulfate Form VI or Form VII.

A ninth aspect of the present invention is to provide crystalline mitapivat hemi sulfate Form IX.

Other aspect of the present invention is to provide a process for the preparation of crystalline mitapivat hemi sulfate Form IX comprising the steps of:
a) suspending mitapivat in a mixture of isobutyl acetate and trifluoro ethanol;
b) adding sulfuric acid diluted with isobutyl acetate to the reaction mixture obtained in step (a);
c) heating the reaction mixture to 40-50° C.; and
d) isolating the crystalline mitapivat hemi sulfate Form IX.

A tenth aspect of the present invention is to provide crystalline mitapivat hemi sulfate Form X.

Other aspect of the present invention is to provide a process for the preparation of crystalline mitapivat hemi sulfate Form X comprising the steps of:
a) suspending mitapivat in a mixture of isobutyl acetate and trifluoro ethanol;
b) adding sulfuric acid diluted with isobutyl acetate to the reaction mixture obtained in step (a);
c) heating the reaction mixture to 40-50° C.;
d) cooling the reaction mixture to 20-30° C.; and
e) isolating the crystalline mitapivat hemi sulfate Form X.

An eleventh aspect of the present invention is to provide crystalline mitapivat hemi sulfate Form XI.

Other aspect of the present invention is to provide a process for the preparation of crystalline mitapivat hemi sulfate Form XI comprising drying crystalline mitapivat hemi sulfate Form IX or Form X or Form XII.

A twelfth aspect of the present invention is to provide crystalline mitapivat hemi sulfate Form XII.

A thirteenth aspect of the present invention is to provide crystalline mitapivat Form I.

Another aspect of the present invention is to provide a process for the preparation of crystalline mitapivat Form I, comprising the steps of:
a) providing mitapivat in organic solvent;
b) cooling the reaction mixture to −5 to +5° C.; and
c) isolating crystalline mitapivat Form I.

A fourteenth aspect of the present invention is to provide crystalline mitapivat Form II.

Other aspect of the present invention is to provide a process for the preparation of crystalline mitapivat Form II comprising the steps of:
a) dissolving mitapivat in an organic solvent at elevated temperature;
b) cooling the reaction mass to 20-30° C.;
c) adding an anti-solvent to the reaction mixture obtained in step (b); and
d) isolating crystalline mitapivat Form II.

A fifteenth aspect of the present invention is to provide crystalline mitapivat Form III.

Other aspect of the present invention is to provide a process for the preparation of crystalline mitapivat Form III comprising the steps of:
a) dissolving mitapivat in acetic acid;
b) adding organic solvent and heating the reaction mixture; and
c) isolating crystalline mitapivat Form III.

A sixteenth aspect of the present invention is to provide crystalline mitapivat Form IV.

A seventeenth aspect of the present invention is to provide crystalline mitapivat mono sulfate Form I.

Another aspect of the present invention is to provide a process for the preparation of crystalline mitapivat mono sulfate Form I, comprising the steps of:
a) dissolving mitapivat in acetic acid;
b) diluting sulfuric acid in acetic acid and adding to the reaction mixture obtained in step (a);
c) optionally seeding with crystalline mitapivat mono sulfate Form I; and
d) isolating crystalline mitapivat mono sulfate Form I.

An eighteenth aspect of the present invention is to provide crystalline mitapivat mono sulfate Form II.

Other aspect of the present disclosure provides a process for the preparation of crystalline mitapivat mono sulfate Form II by treating crystalline mitapivat mono sulfate Form I in humidity.

Another aspect of the present invention is to provide a process for the preparation of crystalline mitapivat mono sulfate Form II comprising the steps of:
a) providing crystalline mitapivat mono sulfate Form I in a mixture of water and organic solvent; and
b) isolating crystalline mitapivat mono sulfate Form II.

A nineteenth aspect of the present invention is to provide crystalline mitapivat mono sulfate Form III.

Other aspect of the present disclosure provides a process for the preparation of crystalline mitapivat mono sulfate Form III by drying crystalline mitapivat mono sulfate Form I.

Another aspect of the present invention is to provide a process for the preparation of crystalline mitapivat mono sulfate Form III comprising the steps of:
a) providing crystalline mitapivat mono sulfate Form I in an organic solvent;
b) heating the reaction mixture; and
c) isolating crystalline mitapivat mono sulfate Form III.

A twentieth aspect of the present invention is to provide crystalline mitapivat mono sulfate Form IV.

Other aspect of the present disclosure provides a process for the preparation of crystalline mitapivat mono sulfate Form IV by drying crystalline mitapivat mono sulfate Form II.

Another aspect of the present invention is to provide a process for the preparation of crystalline mitapivat mono sulfate Form IV comprising the steps of:
a) providing crystalline mitapivat mono sulfate Form II in an organic solvent;
b) heating the reaction mixture; and
c) isolating crystalline mitapivat mono sulfate Form IV.

A twenty first aspect of the present invention is to provide crystalline mitapivat mono sulfate Form V.

Other aspect of the present invention is to provide a process for the preparation of crystalline mitapivat mono sulfate Form V comprising the steps of:
a) suspending mitapivat mono sulfate in a mixture of water and organic solvent;
b) heating and adding water; and
c) isolating crystalline mitapivat mono sulfate Form V.

A twenty second aspect of the present invention is to provide amorphous form of mitapivat mono sulfate.

Other aspect of the present invention is to provide a process for the preparation of amorphous mitapivat mono sulfate comprising the steps of:
a) dissolving mitapivat in polar solvent;
b) adding sulfuric acid diluted water; and
c) lyophilizing to get amorphous mitapivat mono sulfate.

A twenty third aspect of the present invention is to provide a process for the preparation of mitapivat of formula I, which comprising the step of condensing the compound of formula III with 8-quinoline sulfonyl chloride (II).

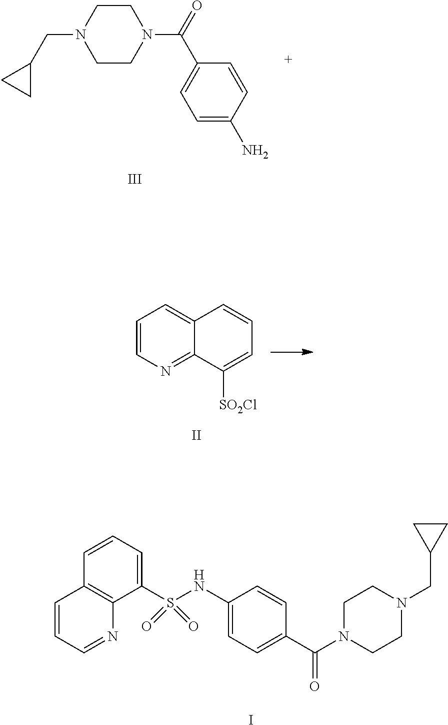

A twenty fourth aspect of the present invention is to provide a process for the preparation of compound of formula III,

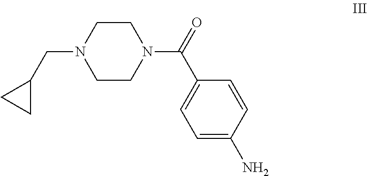 [SMILES: Nc1ccc(C(=O)N2CCN(CC3CC3)CC2)cc1]

which comprises:
   a) condensing cyclopropane piperazine of formula VI or its hydrochloride salt with 4-nitro benzoic acid of formula V to give the compound of formula IV; and

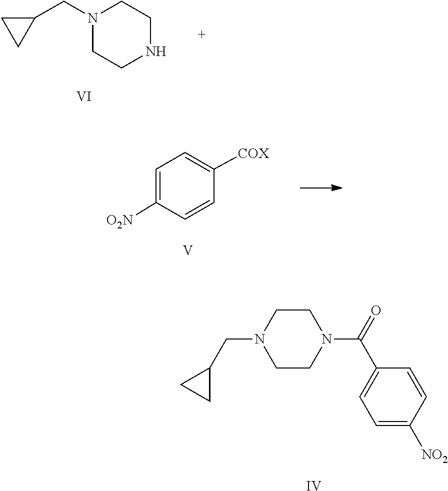

b) reducing the compound of formula IV to obtain the compound of formula III.

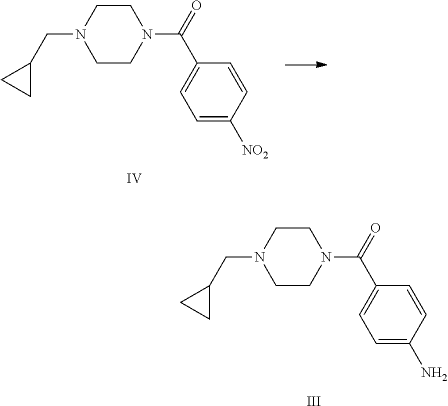

wherein X is selected from —OH or halogen (—Cl of —Br).

### BRIEF DESCRIPTION OF THE FIGURES

Further aspects of the present disclosure together with additional features contributing thereto and advantages accruing there from will be apparent from the following description of embodiments of the disclosure which are shown in the accompanying drawing figures wherein:

FIG. 1 is an X-ray powder diffractogram of crystalline mitapivat hemi sulfate Form I.

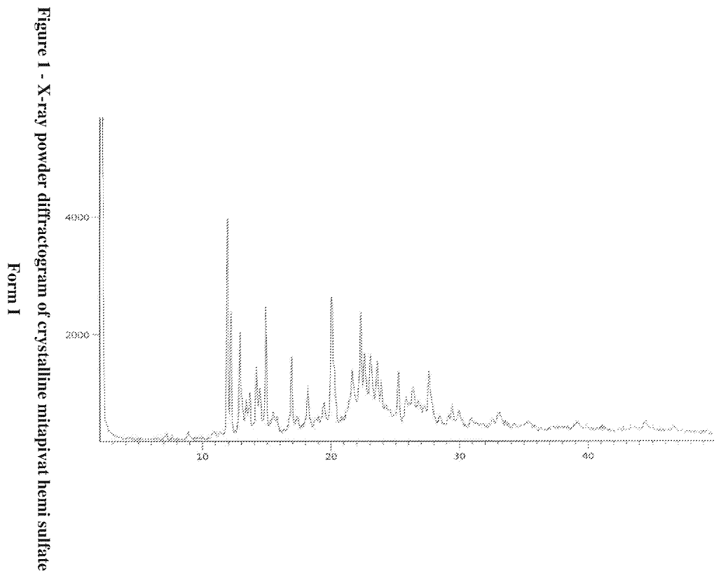

FIG. 2 is an X-ray powder diffractogram of crystalline mitapivat hemi sulfate Form II.

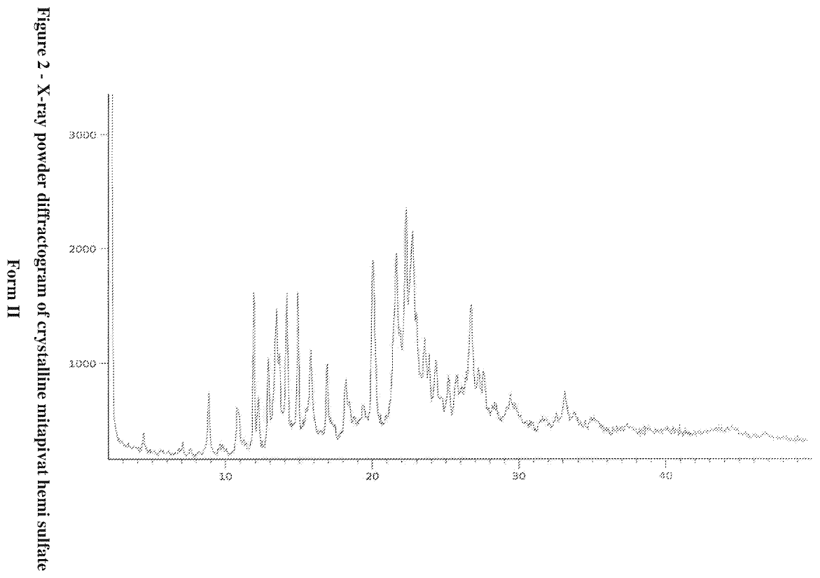

FIG. 3 is an X-ray powder diffractogram of crystalline mitapivat hemi sulfate Form III.

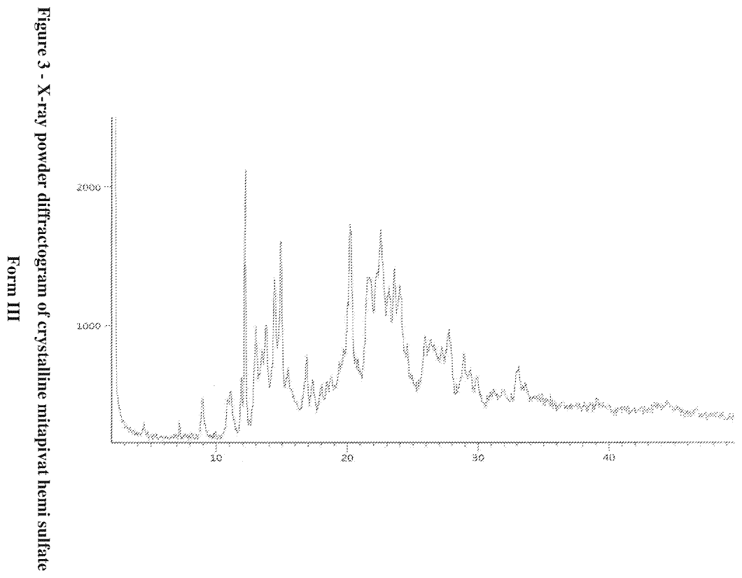

FIG. 4 is an X-ray powder diffractogram of crystalline mitapivat hemi sulfate Form IV.

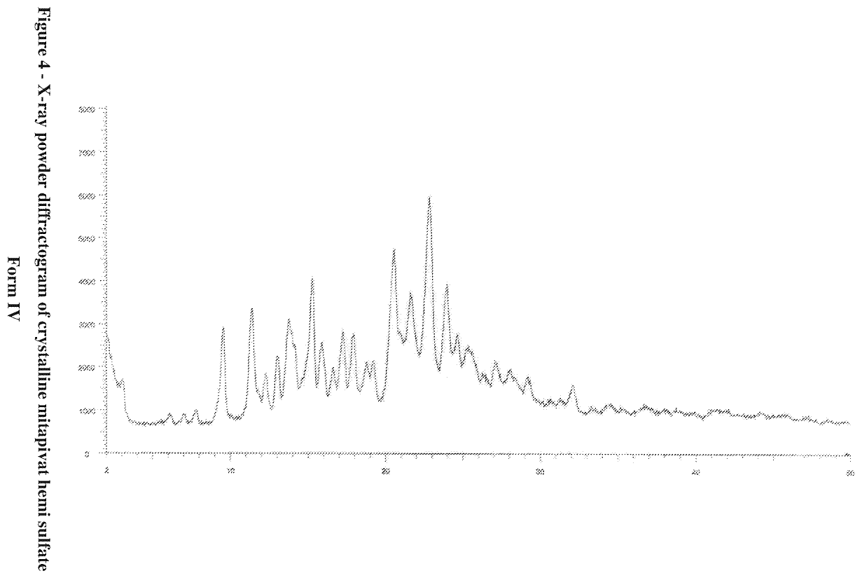

FIG. 5 is an X-ray powder diffractogram of crystalline mitapivat hemi sulfate Form V.

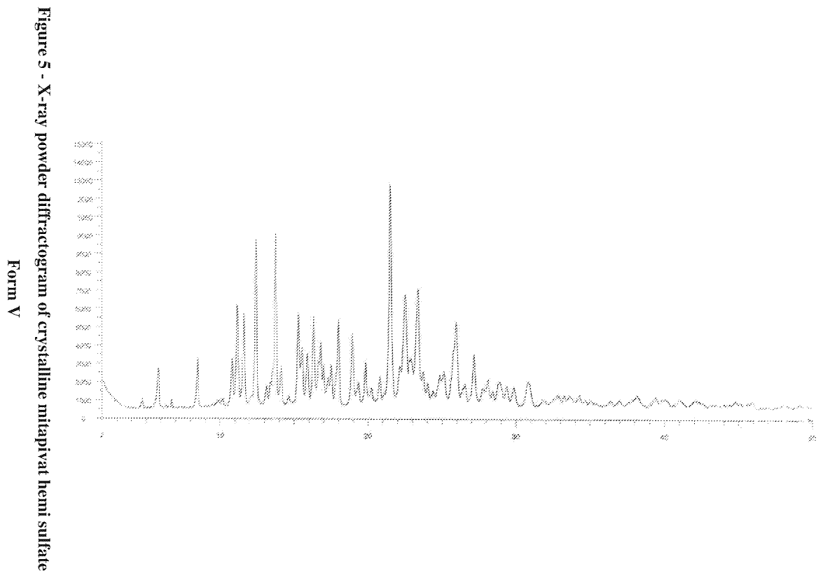

FIG. 6 is an X-ray powder diffractogram of crystalline mitapivat hemi sulfate Form VI.

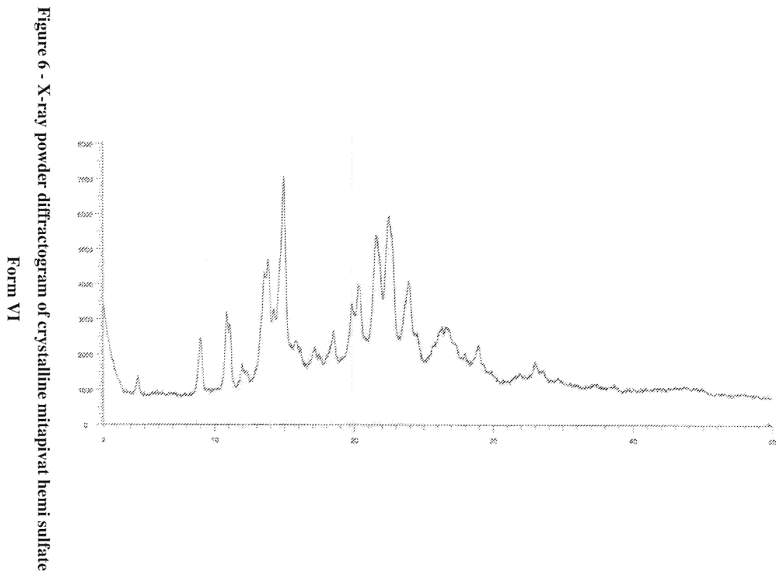

FIG. 7 is an X-ray powder diffractogram of crystalline mitapivat hemi sulfate Form VII.

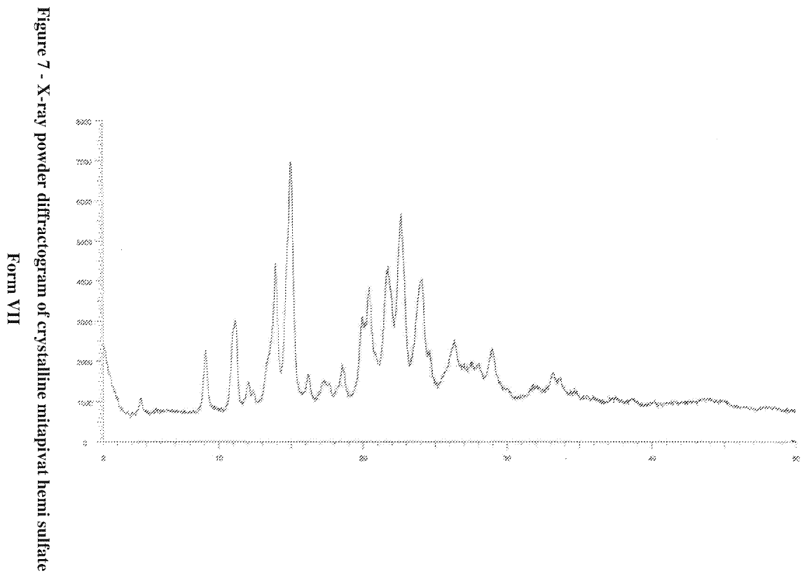

FIG. 8 is an X-ray powder diffractogram of crystalline mitapivat hemi sulfate Form VIII.

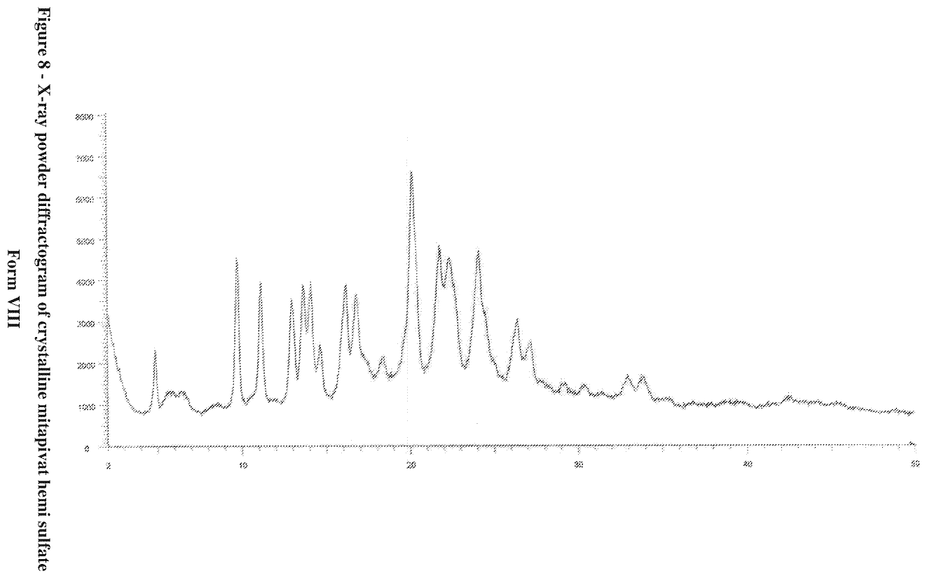

FIG. 9 is an X-ray powder diffractogram of crystalline mitapivat hemi sulfate Form IX.

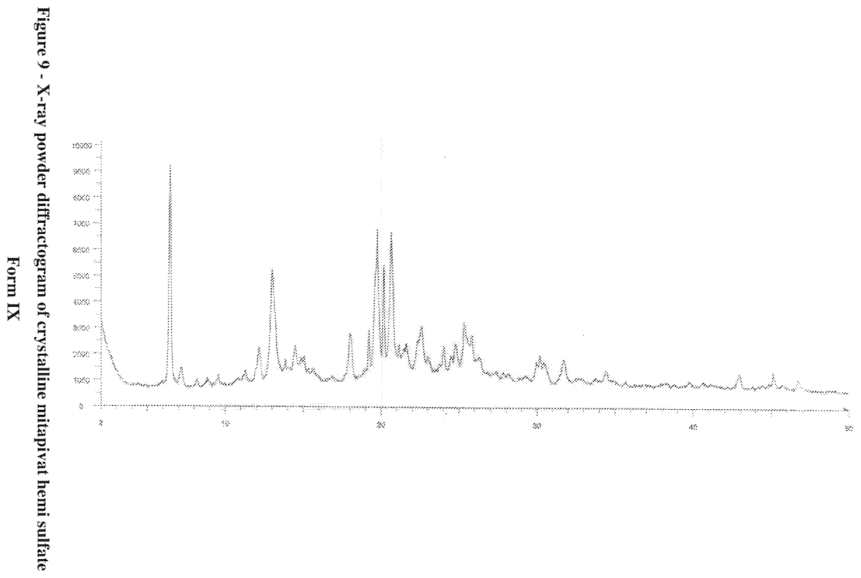

FIG. 10 is an X-ray powder diffractogram of crystalline mitapivat hemi sulfate Form X.

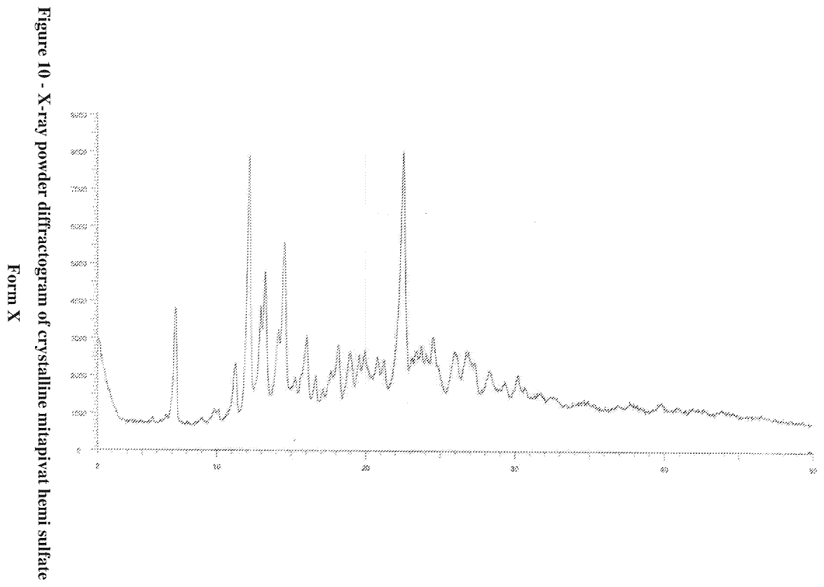

FIG. 11 is an X-ray powder diffractogram of crystalline mitapivat hemi sulfate Form XI.

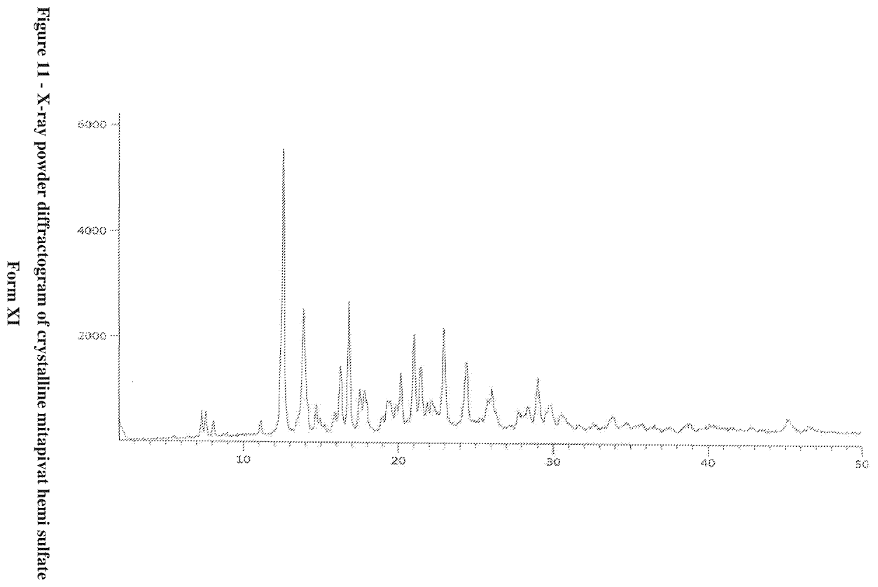

FIG. 12 is an X-ray powder diffractogram of crystalline mitapivat hemi sulfate Form XII.

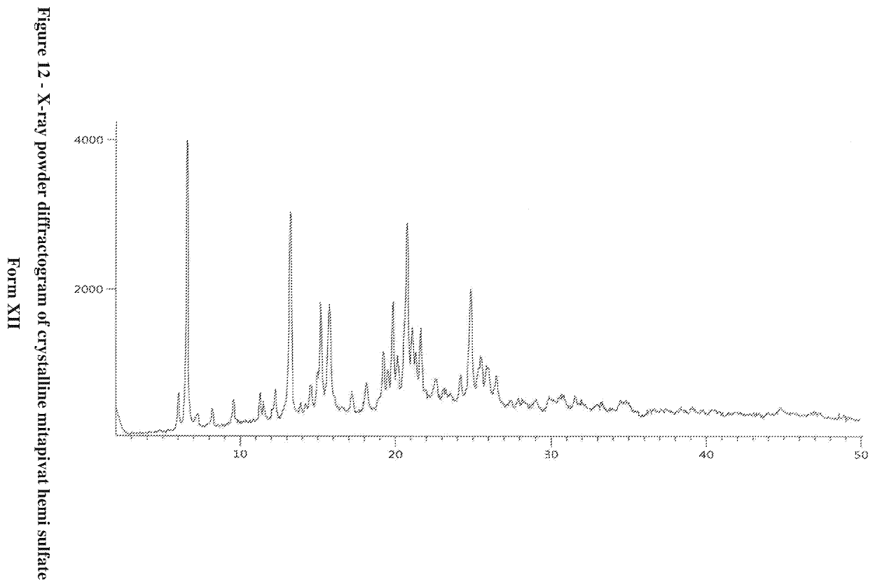

FIG. 13 is an X-ray powder diffractogram of crystalline mitapivat Form I.

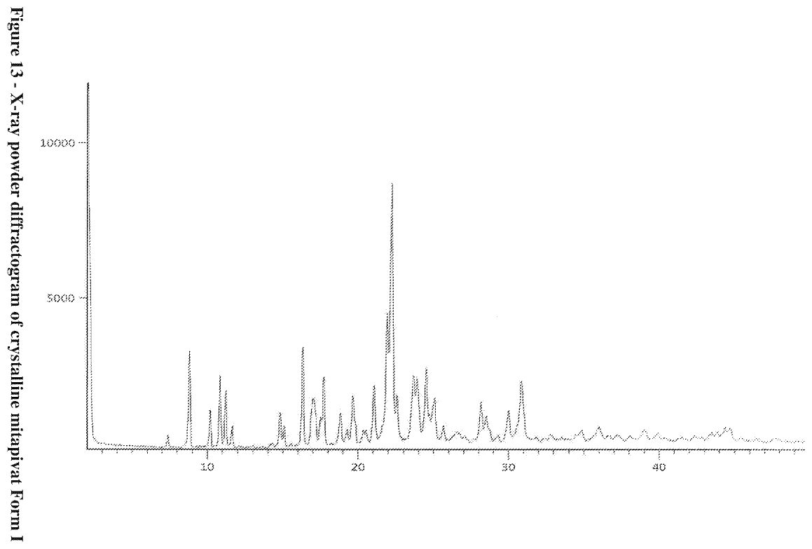

FIG. 14 is an X-ray powder diffractogram of crystalline mitapivat Form II.

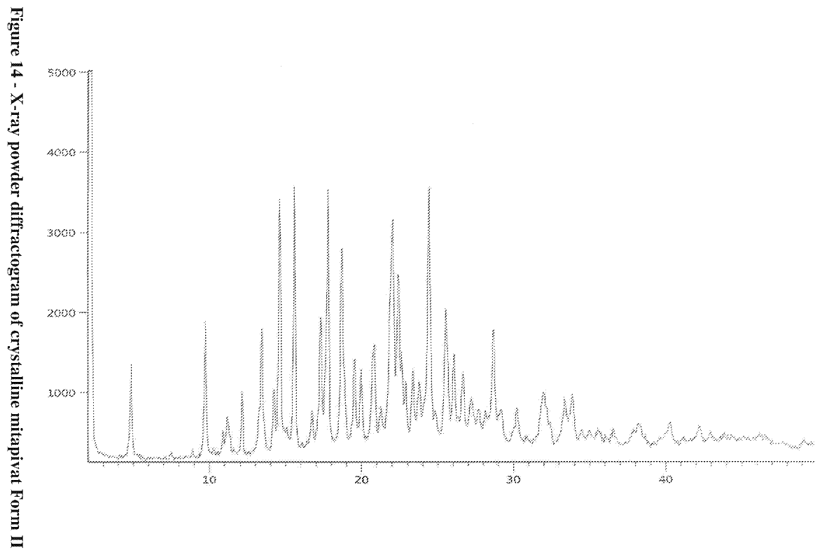

FIG. 15 is an X-ray powder diffractogram of crystalline mitapivat Form III.

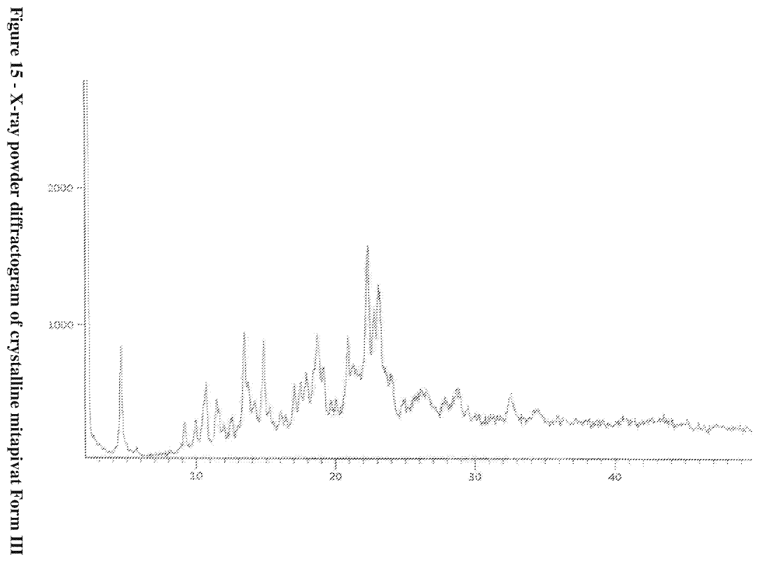

FIG. 16 is an X-ray powder diffractogram of crystalline mitapivat Form IV.

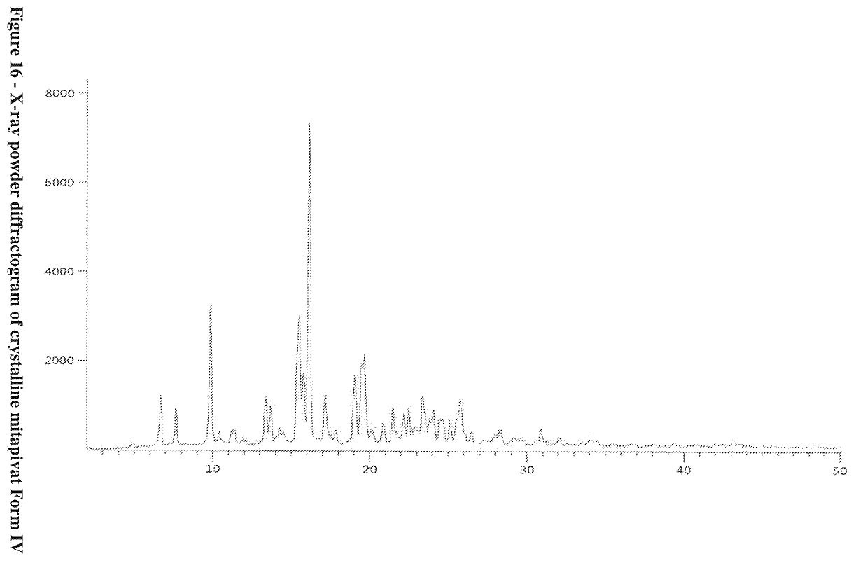

FIG. 17 is an X-ray powder diffractogram of crystalline mitapivat mono sulfate Form I.

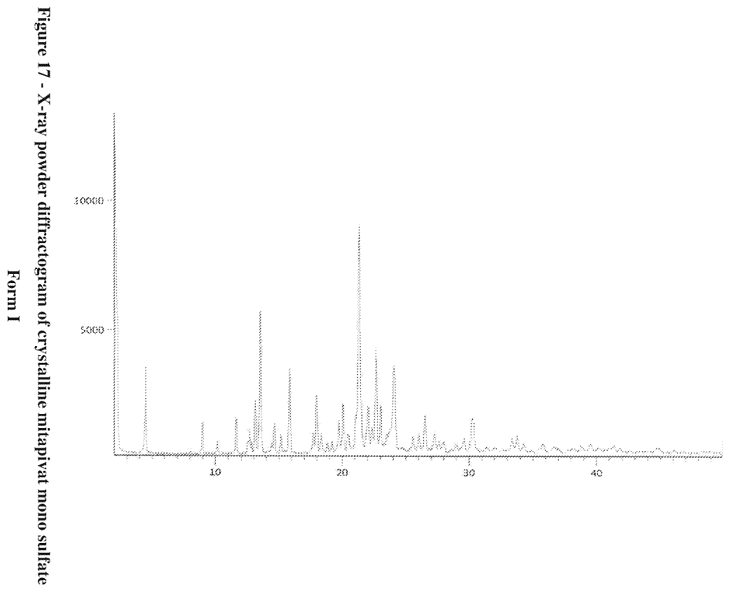

FIG. 18 is an X-ray powder diffractogram of crystalline mitapivat mono sulfate Form II.

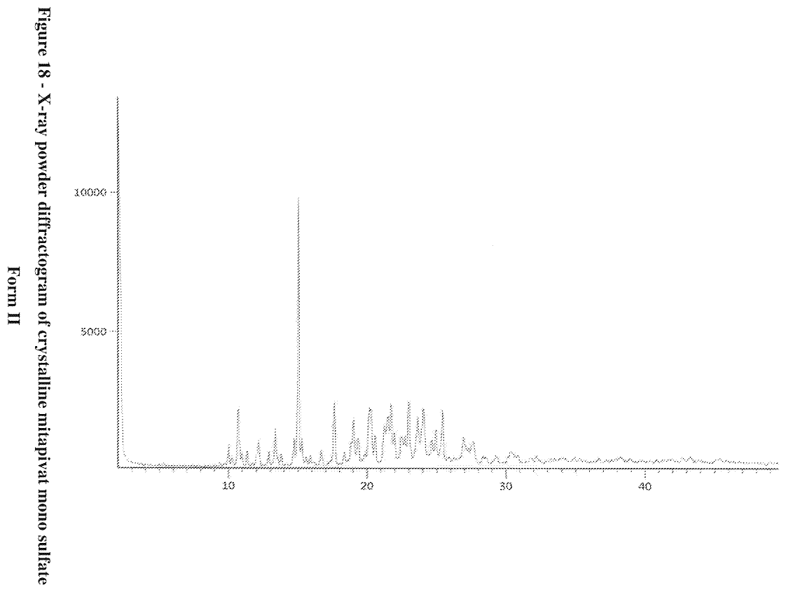

FIG. 19 is an X-ray powder diffractogram of crystalline mitapivat mono sulfate Form III.

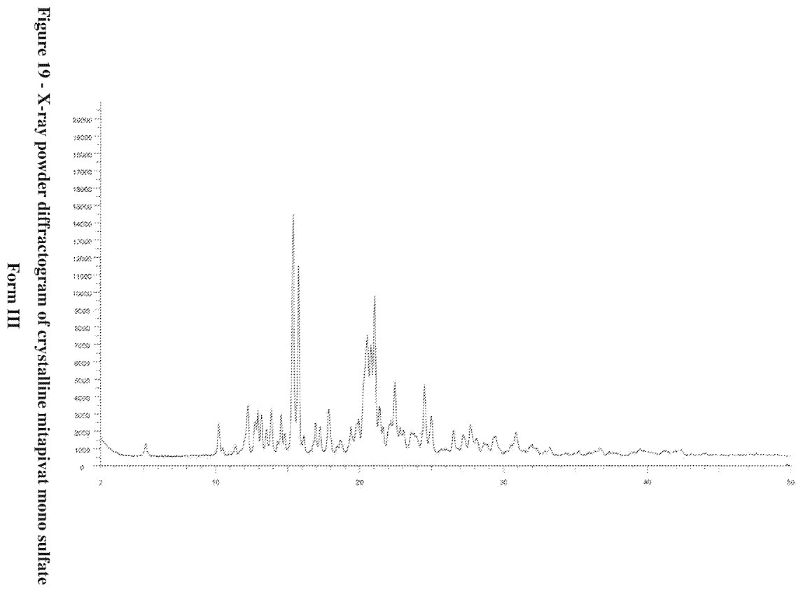

FIG. 20 is an X-ray powder diffractogram of crystalline mitapivat mono sulfate Form IV.

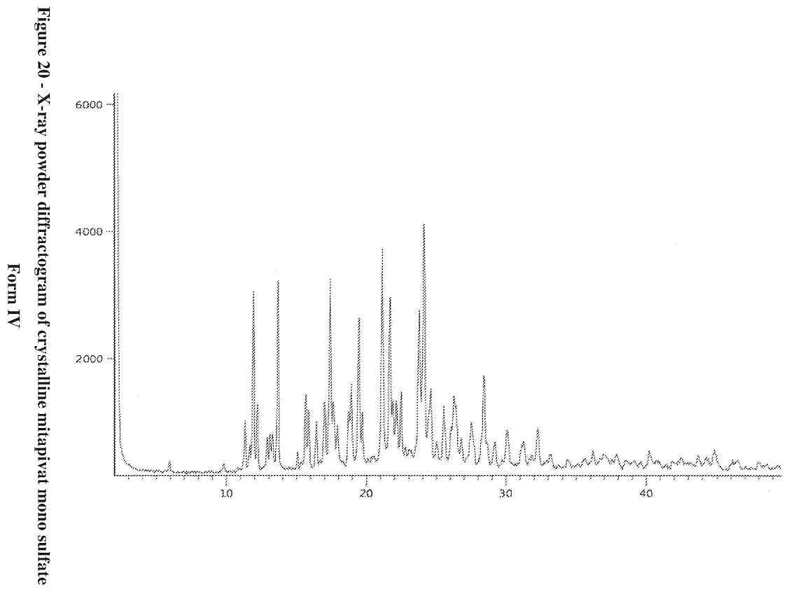

FIG. 21 is an X-ray powder diffractogram of crystalline mitapivat mono sulfate Form V.

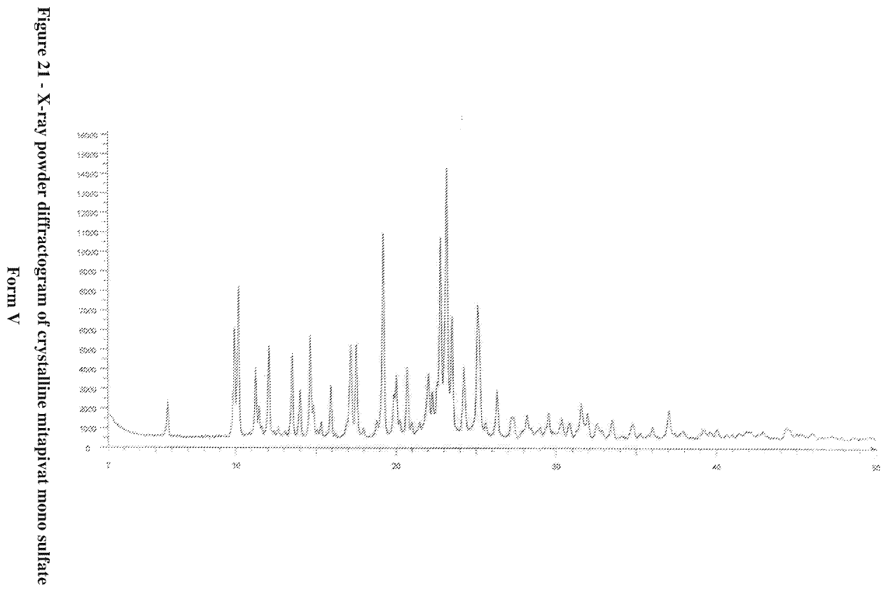

FIG. 22 is an X-ray powder diffractogram of amorphous mitapivat mono sulfate.

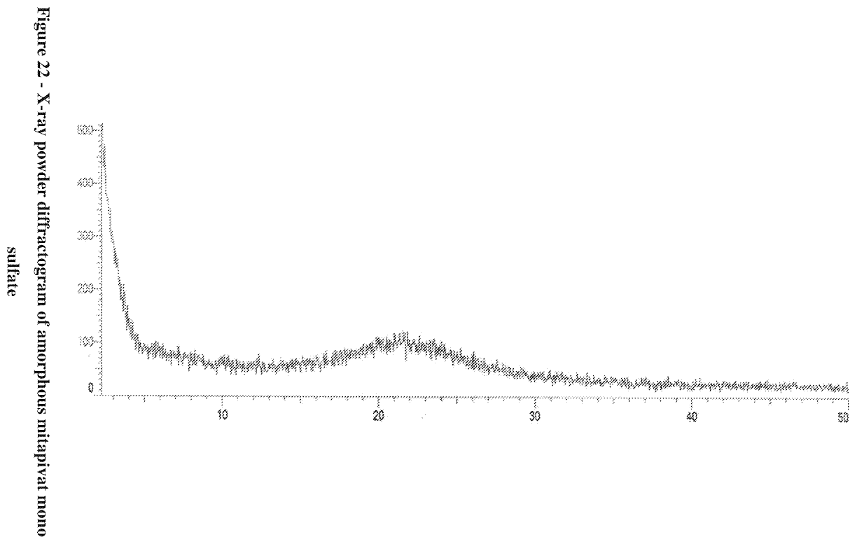

### DETAILED DESCRIPTION OF THE DISCLOSURE

It is to be understood that the description of the present invention has been simplified to illustrate elements that are relevant for a clear understanding of the invention, while eliminating, for purposes of clarity, other elements that may be well known.

The powder X-ray diffraction patterns of said polymorphs of the invention were measured on a PANalytical X′Pert PRO powder diffractometer equipped with a goniometer of θ/θ configuration and X'Celerator detector. The Cu-anode X-ray tube was operated at 40 kV and 30 mA. The experiments were conducted over the 2θ range of 2.0°-50.0°, 0.0330 step size and 50 seconds step time.

Within the context of the invention, the term “about” when modifying an absolute measurement, such as time, mass, or volume, is meant to mean the recited value plus or minus 10% of that value. Within the context of the invention, the term “about” when modifying a temperature measurement is meant to mean the recited temperature plus or minus five degrees.

The present disclosure relates to crystalline forms of mitapivat, mitapivat hemi sulfate and mitapivat mono sulfate. The present disclosure also relates to process for the preparation of mitapivat, crystalline forms of mitapivat, mitapivat hemi sulfate and mitapivat mono sulfate. The present disclosure also relates to amorphous mitapivat mono sulfate.

In one embodiment, the present disclosure is to provide crystalline mitapivat hemi sulfate Form I.

Another embodiment, the present disclosure is to provide crystalline mitapivat hemi sulfate Form I, characterized by Powder X-ray diffraction pattern having 20 angle positions at about 11.93, 12.90, 14.92, 20.04 and 22.31±0.2° degrees two-theta.

In yet another embodiment, crystalline mitapivat hemi sulfate Form I is further characterized by Powder X-ray diffraction pattern having 2θ angle positions at about 11.93, 12.21, 12.90, 14.18, 14.45, 14.92, 16.92, 18.19, 20.04, 21.63, 22.31, 22.58, 23.05, 23.58, 23.90, 25.25 and 27.61±0.2° degrees two-theta.

In still another embodiment the present invention is to provide a process for the preparation of crystalline mitapivat hemi sulfate Form I, comprising the steps of:
a) providing mitapivat in a mixture of solvents and adding sulfuric acid;
b) heating the reaction mixture;
c) optionally reducing the volume of the reaction mass by removing the solvent;
d) adding organic solvent to the reaction mixture; and
e) isolating crystalline mitapivat hemi sulfate Form I.

Within the context of this embodiment, the mixture of solvents employed may include mixture of alcohols and ester solvents, for example, but not limited ethanol, methanol, n-propanol, n-butanol, 2-butanol, 2-propanol, 3-methyl-1-butanol, 1-pentanol, 2-methyl-1-propanol, ethyl acetate, n-propyl acetate, isopropyl acetate, n-butyl acetate, isobutyl acetate, methyl acetate. In particular useful embodiments mixture of methanol and isobutyl acetate is used.

Within the context of this embodiment, sulfuric acid added is concentrated or diluted with same or different solvents used in the above step.

Within the context of this embodiment, organic solvent employed in step (d) may include alcohols, esters and ketones solvents, for example, but not limited to ethanol, methanol, n-propanol, n-butanol, 2-butanol, 2-propanol, 3-methyl-1-butanol, 1-pentanol, 2-methyl-1-propanol, ethyl acetate, n-propyl acetate, isopropyl acetate, n-butyl acetate, isobutyl acetate, methyl acetate, acetone, methyl ethyl ketone, methyl isobutyl ketone, methyl butyl ketone. In particular useful embodiments methanol and isobutyl acetate is used.

Within the context of this embodiment, addition of organic solvent in step (d) can be done in multiple times with same or different solvents as used in step (a).

Within the context of this embodiment, isolation can be done using any techniques in the art such as, decantation, filtration by gravity or suction, centrifugation. In particular useful embodiments the solid is isolated by filtration followed by suck-drying under vacuum.

According to the present disclosure, mitapivat is suspended in mixture of solvents, added sulfuric acid solution, and heated to elevated temperature of about 50° C. to boiling point of the solvent. The volume of the reaction mass was reduced and then diluted with organic solvents. The reaction mixture was cooled to about 2-8° C. The obtained solid is filtered, washed with organic solvent and then suck-dried, which was identified as crystalline mitapivat hemi sulfate Form I.

In another embodiment, the present disclosure is to provide crystalline mitapivat hemi sulfate Form II.

Another embodiment, the present disclosure is to provide crystalline mitapivat hemi sulfate Form II, characterized by Powder X-ray diffraction pattern having 20 angle positions at about 13.46, 20.03, 21.64, 22.29 and 22.75±0.2° degrees two-theta.

In yet another embodiment, crystalline mitapivat hemi sulfate Form II is further characterized by Powder X-ray diffraction pattern having 2θ angle positions at about 8.86, 11.92, 12.23, 12.90, 13.46, 13.68, 14.17, 14.92, 15.80, 16.92, 18.18, 20.03, 21.64, 22.29, 22.75, 23.57, 23.89, 24.32, 25.78, 26.73, 27.25 and 27.59±0.2° degrees two-theta.

In still another embodiment the present invention is to provide a process for the preparation of crystalline mitapivat hemi sulfate Form II comprising the steps of:
a) providing mitapivat in a mixture of solvents and adding sulfuric acid;
b) heating the reaction mixture;
c) reducing the volume of the reaction mass by removing the solvent;
d) cooling the reaction mixture obtained in step (c); and
e) isolating crystalline mitapivat hemi sulfate Form II.

Within the context of this embodiment, the mixture of solvents employed may include mixture of alcohols and ester solvents, for example, but not limited ethanol, methanol, n-propanol, n-butanol, 2-butanol, 2-propanol, 3-methyl-1-butanol, 1-pentanol, 2-methyl-1-propanol, ethyl acetate, n-propyl acetate, isopropyl acetate, n-butyl acetate, isobutyl acetate, methyl acetate. In particular useful embodiments mixture of methanol and isobutyl acetate is used.

Within the context of this embodiment, sulfuric acid added is concentrated or diluted with same or different solvents used in the above step.

Within the context of this embodiment, isolation can be done using any techniques in the art such as, decantation, filtration by gravity or suction, centrifugation. In particular useful embodiments the solid is isolated by filtration followed by suck-drying under vacuum.

According to the present disclosure, mitapivat is suspended in mixture of solvents, added sulfuric acid solution and heated to elevated temperature of about 50° C. to boiling point of the solvent. The volume of the reaction mass was reduced and then cooled to about 2-8° C. The obtained solid is filtered, washed with solvent and then suck-dried, which was identified as crystalline mitapivat hemi sulfate Form II.

In another embodiment, the present disclosure is to provide crystalline mitapivat hemi sulfate Form III.

Another embodiment, the present disclosure is to provide crystalline mitapivat hemi sulfate Form III, characterized by Powder X-ray diffraction pattern having 2θ angle positions at about 11.91, 13.81, 15.44, 21.49 and 24.05±0.2° degrees two-theta.

In yet another embodiment, crystalline mitapivat hemi sulfate Form III is further characterized by Powder X-ray diffraction pattern having 2θ angle positions at about 11.91, 12.20, 13.04, 13.81, 14.45, 14.93, 15.44, 16.90, 20.26, 21.49, 22.52, 23.22, 23.58, 24.05, 25.91 and 27.82±0.2° degrees two-theta.

In still another embodiment the present invention is to provide a process for the preparation of crystalline mitapivat hemi sulfate Form III comprising the steps of:
a) providing mitapivat in a mixture of solvents and adding sulfuric acid;
b) heating the reaction mixture obtained in step (a);
c) reducing the reaction mass by removing the solvent; and
d) cooling the reaction mixture obtained in step (c);
e) isolating and drying to get crystalline mitapivat hemi sulfate Form III.

Within the context of this embodiment, the mixture of solvents employed may include mixture of alcohols and ester solvents, for example, but not limited ethanol, methanol, n-propanol, n-butanol, 2-butanol, 2-propanol, 3-methyl-1-butanol, 1-pentanol, 2-methyl-1-propanol, ethyl acetate, n-propyl acetate, isopropyl acetate, n-butyl acetate, isobutyl acetate, methyl acetate. In particular useful embodiments mixture of methanol and isobutyl acetate is used.

Within the context of this embodiment, sulfuric acid added is concentrated or diluted with same or different solvents used in the above step.

Within the context of this embodiment, drying is performed under atmospheric pressure or under reduced pressure. In particular useful embodiments drying is performed under reduced pressure.

According to the present disclosure, mitapivat is suspended in mixture of solvents, added sulfuric acid solution and heated to elevated temperature of about 50° C. to boiling point of the solvent. The volume of the reaction mass was reduced and then cooled to about 5-15° C. The obtained solid is filtered, washed with solvent and then dried under vacuum, which was identified as crystalline mitapivat hemi sulfate Form III.

In still another embodiment the present invention is to provide a process for the preparation of crystalline mitapivat hemi sulfate Form III by drying crystalline mitapivat hemi sulfate Form II under reduced pressure at 30-50° C. for 10-20 hours.

In one embodiment, the present disclosure is to provide crystalline mitapivat hemi sulfate Form IV.

In another embodiment, the present disclosure is to provide crystalline mitapivat hemi sulfate Form IV, characterized by Powder X-ray diffraction pattern having 2θ angle positions at about 9.51, 11.35, 15.25, 20.52, 21.61, 22.84 and 23.95±0.2° degrees two-theta.

In yet another embodiment, crystalline mitapivat hemi sulfate Form IV is further characterized by Powder X-ray diffraction pattern having 2θ angle positions at about 3.00, 9.51, 11.35, 12.25, 13.01, 13.76, 14.08, 15.25, 15.87, 16.58, 17.22, 17.90, 18.76, 19.19, 20.52, 21.64, 22.84, 23.95, 24.64, 25.29, 25.89, 27.13, 28.07, 29.18, 30.66, 32.15 and 32.15±0.2° degrees two-theta.

In still another embodiment the present invention is to provide a process for the preparation of crystalline mitapivat hemi sulfate Form IV comprising the steps of:
a) dissolving mitapivat in acetic acid;
b) adding sulfuric acid diluted with acetic acid;
c) adding an organic solvent to the reaction mixture obtained in step (b); and
d) isolating crystalline mitapivat hemi sulfate Form IV.

Within the context of this embodiment, the organic solvent employed may include esters for example, but not limited to ethyl acetate, isopropyl acetate, isobutyl acetate. In particular useful embodiments organic solvent is ethyl acetate.

Within the context of this embodiment, isolation can be done using any techniques in the art such as, decantation, filtration by gravity or suction, centrifugation. In particular useful embodiments the solid is isolated by filtration followed by suck-drying.

According to the present disclosure, mitapivat is dissolved in an acetic acid at a temperature of 50-70° C. The reaction mass is cooled to 20-30° C. and added sulfuric acid, which is diluted with acetic acid. The reaction mass is stirred for about 14-16 hours and added organic solvent. The resulted reaction mass is stirred, filtered and then suck-dried. The obtained solid was identified as crystalline mitapivat hemi sulfate Form IV.

In another embodiment, the present disclosure provides a process for the preparation of crystalline mitapivat hemi sulfate Form IV comprising the steps of:
a) dissolving mitapivat hemi sulfate in acetic acid;
b) adding an organic solvent; and
c) isolating crystalline mitapivat hemi sulfate Form IV.

Within the context of this embodiment, the organic solvent employed may include esters for example, but not limited to ethyl acetate, isopropyl acetate, isobutyl acetate. In particular useful embodiments organic solvent is ethyl acetate.

Within the context of this embodiment, isolation can be done using any techniques in the art such as, decantation, filtration by gravity or suction, centrifugation. In particular useful embodiments the solid is isolated by filtration followed by suck-drying.

According to the present disclosure, mitapivat hemi sulfate is dissolved in an acetic acid at a temperature of 60-80° C. The reaction mass is cooled to 20-30° C. and added organic solvent. The resulted reaction mass is stirred, filtered, washed with organic solvent and then suck-dried. The obtained solid was identified as crystalline mitapivat hemi sulfate Form IV.

Another embodiment of the present disclosure is to provide crystalline mitapivat hemi sulfate Form V.

In another embodiment, the present disclosure is to provide crystalline mitapivat hemi sulfate Form V, characterized by Powder X-ray diffraction pattern having 2θ angle positions at about 8.45, 11.13, 12.38, 13.72, 21.51, 22.52, 23.37 and 25.95±0.2° degrees two-theta.

Another embodiment, the present disclosure is to provide crystalline mitapivat hemi sulfate Form V, characterized by Powder X-ray diffraction pattern having 2θ angle positions at about 5.76, 8.45, 10.78, 11.13, 11.55, 12.38, 13.10, 13.72, 14.09, 15.28, 15.52, 15.85, 16.28, 16.76, 17.47, 17.97, 18.92, 19.82, 21.51, 22.16, 22.52, 22.87, 23.37, 25.95 and 27.16±0.2° degrees two-theta.

In still another embodiment the present invention is to provide a process for the preparation of crystalline mitapivat hemi sulfate Form V comprising the steps of:
a) suspending crystalline mitapivat hemi sulfate Form IV in a polar solvent; and
b) isolating crystalline mitapivat hemi sulfate Form V.

Within the context of this embodiment, the polar solvent employed may include water, acetone, acetonitrile, methanol, ethanol, ethyl acetate and mixtures thereof. In particular useful embodiments polar solvent is mixture of water and ethyl acetate.

Within the context of this embodiment, isolation can be done using any techniques in the art such as, decantation, filtration by gravity or suction, centrifugation. In particular useful embodiments the solid is isolated by filtration followed by suck-drying under vacuum.

According to the present disclosure, mitapivat hemi sulfate Form IV is suspended in polar solvent, heated to a temperature of about 55-70° C. and then stirred for about 2-3 hours at the same temperature. The reaction mass is filtered, suck dried for 10-15 minutes at 25-30° C. The obtained solid is dried under vacuum at 100° C. for about 8-10 hours. The resulted compound was identified as crystalline mitapivat hemi sulfate Form V.

In another embodiment, the present disclosure is to provide crystalline mitapivat hemi sulfate Form VI.

Another embodiment, the present disclosure is to provide crystalline mitapivat hemi sulfate Form VI, characterized by Powder X-ray diffraction pattern having 2θ angle positions at about 4.45, 8.96, 10.84, 13.57, 13.81, 14.93 and 21.60±0.2° degrees two-theta.

In yet another embodiment, crystalline mitapivat hemi sulfate Form VI is further characterized by Powder X-ray diffraction pattern having 2θ angle positions at about 4.45, 8.96, 10.84, 11.04, 11.95, 12.27, 13.57, 13.81, 14.23, 14.93, 15.81, 17.15, 18.45, 19.83, 20.35, 21.60, 22.52, 23.96, 24.47, 26.32, 26.65, 27.99, 28.98, 31.95 and 33.05±0.2° degrees two-theta.

In still another embodiment the present invention is to provide a process for the preparation of crystalline mitapivat hemi sulfate Form VI comprising the steps of:
a) dissolving mitapivat hemi sulfate in a halogenated alcohol solvent;
b) adding water to the above reaction mixture; and
c) isolating crystalline mitapivat hemi sulfate Form VI.

Within the context of this embodiment, halogenated alcohol solvent, for example, but not limited 2-chloroethanol, 2,2,2-trifluoromethanol, 2-mercaptoethanol, trifluoroethanol, hexafluoro-2-propanol. In particular useful embodiments trifluoroethanol is used.

Within the context of this embodiment, isolation can be done using any techniques in the art such as, decantation, filtration by gravity or suction, centrifugation. In particular useful embodiments the solid is isolated by filtration followed by suck-drying.

According to the present disclosure, mitapivat hemi sulfate is suspended in halogenated alcohol solvent, heated to elevated temperature of about 60° C. to boiling point of the solvent. The reaction mixture was cooled to about 20-30° C. and added water. The reaction mixture is stirred for 2-3 days, the obtained solid is filtered, washed with water and then suck-dried, which was identified as crystalline mitapivat hemi sulfate Form VI.

In another embodiment the present invention is to provide a process for the preparation of crystalline mitapivat hemi sulfate Form VI comprising the steps of:
a) diluting sulfuric acid with water and adding the seeds of crystalline mitapivat hemi sulfate Form VI to the diluted sulfuric acid solution;
b) dissolving mitapivat in a halogenated alcohol solvent and adding to the reaction mixture obtained in step (a) or vice-versa; and
c) isolating crystalline mitapivat hemi sulfate Form VI.

Within the context of this embodiment, halogenated alcohol solvent, for example, but not limited 2-chloroethanol, 2,2,2-trifluoromethanol, 2-mercaptoethanol, trifluoroethanol, hexafluoro-2-propanol. In particular useful embodiments trifluoroethanol is used.

Within the context of this embodiment, isolation can be done using any techniques in the art such as, decantation, filtration by gravity or suction, centrifugation. In particular useful embodiments the solid is isolated by filtration followed by suck-drying.

According to the present disclosure, to the sulfuric acid solution in water added seeds of mitapivat hemi sulfate Form VI, and then added mitapivat, which is dissolved in halogenated alcohol solvent at 0-8° C. The reaction mixture temperature was then raised to about 20-30° C. and stirred for 6-8 hours. The reaction mixture was cooled to about 0-5° C. and stirred. The solid obtained is filtered and suck-dried, which was identified as crystalline mitapivat hemi sulfate Form VI.

In another embodiment, the present disclosure is to provide crystalline mitapivat hemi sulfate Form VII.

Another embodiment, the present disclosure is to provide crystalline mitapivat hemi sulfate Form VII, characterized by Powder X-ray diffraction pattern having 2θ angle positions at about 9.07, 13.92, 14.99, 20.43, 21.72 and 24.08±0.2° degrees two-theta.

In yet another embodiment, crystalline mitapivat hemi sulfate Form VII is further characterized by Powder X-ray diffraction pattern having 2θ angle positions at about 9.07, 11.14, 12.02, 13.92, 14.99, 16.19, 17.28, 17.61, 18.58, 19.93, 20.43, 21.72, 22.66, 24.08, 24.61, 26.37, 28.04, 29.01, 31.83, 33.22 and 33.66±0.2° degrees two-theta.

In still another embodiment the present invention is to provide a process for the preparation of crystalline mitapivat hemi sulfate Form VII comprising the steps of:
a) dissolving mitapivat hemi sulfate in a halogenated alcohol solvent;
b) reducing the volume of the reaction mixture obtained in step (a);
c) adding water to reaction mixture obtained in step (b);
d) subjecting the reaction mixture obtained in step (C) to sonication; and
e) isolating the crystalline mitapivat hemi sulfate Form VII.

Within the context of this embodiment, halogenated alcohol solvent, for example, but not limited 2-chloroethanol, 2,2,2-trifluoromethanol, 2-mercaptoethanol, trifluoroethanol, hexafluoro-2-propanol. In particular useful embodiments trifluoroethanol is used.

Within the context of this embodiment, isolation can be done using any techniques in the art such as, decantation, filtration by gravity or suction, centrifugation. In particular useful embodiments the solid is isolated by filtration followed by suck-drying.

According to the present disclosure, mitapivat hemi sulfate dissolved in halogenated alcohols at elevated temperature of about 70° C. to boiling point of the solvent. The solution slowly evaporated at 60-80° C. The reaction mixture was cooled to about 20-30° C. and added water. The resulted solution sonicated. The solid obtained is filtered and suck-dried, which was identified as crystalline mitapivat hemi sulfate Form VII.

Other embodiment of the present invention is to provide a process for the preparation of crystalline mitapivat hemi sulfate Form VII comprising the steps of:
a) adding seeds of crystalline mitapivat hemi sulfate Form VII to water;
b) dissolving mitapivat hemi sulfate in a halogenated alcohol solvent and adding to the reaction mixture obtained in step (a) or vice-versa; and
c) isolating crystalline mitapivat hemi sulfate Form VII.

Within the context of this embodiment, halogenated alcohol solvent, for example, but not limited 2-chloroethanol, 2,2,2-trifluoromethanol, 2-mercaptoethanol, trifluoroethanol, hexafluoro-2-propanol. In particular useful embodiments trifluoroethanol is used.

Within the context of this embodiment, isolation can be done using any techniques in the art such as, decantation, filtration by gravity or suction, centrifugation. In particular useful embodiments the solid is isolated by filtration followed by suck-drying.

According to the present disclosure, mitapivat hemi sulfate dissolved in halogenated alcohols at elevated temperature of about 70° C. to boiling point of the solvent. The solution cooled to 20-30° C. In separate flask water cooled to 2-10° C. and added seeds of crystalline mitapivat hemi sulfate Form VII after that added prepared mitapivat hemi sulfate solution. The reaction mass obtained stirred at 2-10° C. for 1-2 hours. The solid obtained is filtered and suck-dried, which was identified as crystalline mitapivat hemi sulfate Form VII.

Another embodiment of the present invention is to provide a process for the preparation of crystalline mitapivat hemi sulfate Form VII comprising the steps of:
a) diluting sulfuric acid with water;
b) dissolving mitapivat in a halogenated alcohol solvent and adding to the sulfuric acid solution obtained in step (a) or vice-versa;
c) adding water to the reaction mixture obtained in step (b); and
d) isolating crystalline mitapivat hemi sulfate Form VII.

Within the context of this embodiment, halogenated alcohol solvent, for example, but not limited 2-chloroethanol, 2,2,2-trifluoromethanol, 2-mercaptoethanol, trifluoroethanol, hexafluoro-2-propanol. In particular useful embodiments trifluoroethanol is used.

Within the context of this embodiment, isolation can be done using any techniques in the art such as, decantation, filtration by gravity or suction, centrifugation. In particular useful embodiments the solid is isolated by filtration followed by suck-drying.

According to the present disclosure, mitapivat dissolved in halogenated alcohols at elevated temperature of about 70° C. to boiling point of the solvent. The solution cooled to 20-30° C. In separate flask added water and sulfuric acid solution diluted with water. Cooled the solution to 2-10° C. and added prepared of mitapivat solution. Added water to the reaction mass, stirred at 2-10° C. for 1-2 hours. The solid obtained is filtered at 2-10° C. and suck-dried for 30-60 minutes at 20-30° C. and then dried under vacuum at 35-45° C. for 65-75 hours. The product obtained, which was identified as crystalline mitapivat hemi sulfate Form VII.

Other embodiment of the present invention is to provide a process for the preparation of crystalline mitapivat hemi sulfate Form VII comprising the steps of:
a) dissolving mitapivat in a halogenated alcohol solvent;
b) adding water to the reaction mixture obtained in step (a) and adding sulfuric acid diluted with water;
c) adding the seeds of crystalline mitapivat hemi sulfate VII; and
d) isolating crystalline mitapivat hemi sulfate Form VII.

Within the context of this embodiment, halogenated alcohol solvent, for example, but not limited 2-chloroethanol, 2,2,2-trifluoromethanol, 2-mercaptoethanol, trifluoroethanol, hexafluoro-2-propanol. In particular useful embodiments trifluoroethanol is used.

Within the context of this embodiment, isolation can be done using any techniques in the art such as, decantation, filtration by gravity or suction, centrifugation. In particular useful embodiments the solid is isolated by filtration followed by suck-drying.

According to the present disclosure, mitapivat dissolved in halogenated alcohols. To the solution added water and cooled to 0-5° C. To the cooled solution added sulfuric acid solution diluted with water followed by seeds of crystalline hemi sulfate Form VII. The reaction mass is stirred for 4-7 hours at 0-5° C. then raised temperature to 20-30° C., stirred for 6-8 hours. The reaction mass is cooled to 0-5° C. and stirred for 1-2 hours. The solid obtained is filtered, washed with water and suck dried. The product obtained, which was identified as crystalline mitapivat hemi sulfate Form VII.

Other embodiment of the present invention is to provide a process for the preparation of crystalline mitapivat hemi sulfate Form VII comprising the steps of:
a) dissolving mitapivat in a halogenated alcohol solvent;
b) adding water to the reaction mixture obtained in step (a) and cooling;
c) optionally adding seeds of crystalline mitapivat hemi sulfate VII; and
d) adding sulfuric acid;
e) isolating crystalline mitapivat hemi sulfate Form VII.

Within the context of this embodiment, halogenated alcohol solvent, for example, but not limited 2-chloroethanol, 2,2,2-trifluoromethanol, 2-mercaptoethanol, trifluoroethanol, hexafluoro-2-propanol. In particular useful embodiments trifluoroethanol is used.

Within the context of this embodiment, sulfuric acid can be diluted with a solvent, preferably with water.

Within the context of this embodiment, isolation can be done using any techniques in the art such as, decantation, filtration by gravity or suction, centrifugation. In particular useful embodiments the solid is isolated by filtration followed by suck-drying.

According to the present disclosure, mitapivat dissolved in halogenated alcohols. To the solution added water and cooled to −10 to 15° C., preferably 0-5° C. To the cooled
solution optionally added seeds of crystalline hemi sulfate Form VII followed by sulfuric acid, which can be diluted with water. The reaction mass is stirred for 22-27 hours at −10 to 15° C., preferably 0-5° C. The solid obtained is filtered, washed with mixture of halogenated alcohol solvent and water, suck dried. The solid obtained is dried at 34-45° C. under vacuum for 15-20 hours. The product obtained, which was identified as crystalline mitapivat hemi sulfate Form VII.

Another embodiment, the present disclosure is to provide crystalline mitapivat hemi sulfate Form VIII, characterized by Powder X-ray diffraction pattern having 2θ angle positions at about 4.81, 11.09, 12.95, 16.11, 20.15 and 24.08±0.2° degrees two-theta.

In yet another embodiment, crystalline mitapivat hemi sulfate Form VIII is further characterized by Powder X-ray diffraction pattern having 2θ angle positions at about 4.81, 6.39, 8.51, 9.71, 11.09, 12.95, 13.64, 14.07, 14.61, 16.11, 16.77, 17.32, 18.33, 20.15, 21.77, 22.34, 24.08, 26.34, 27.15, 29.13, 30.42, 32.99 and 33.88±0.2° degrees two-theta.

In still another embodiment the present invention is to provide a process for the preparation of crystalline mitapivat hemi sulfate Form VIII comprising drying crystalline mitapivat hemi sulfate Form VI or Form VII.

Within the context of this embodiment, drying is performed under atmospheric pressure or under reduced pressure. In particular useful embodiments drying is performed under reduced pressure.

According to the present disclosure, crystalline mitapivat hemi sulfate Form VI or crystalline mitapivat hemi sulfate Form VII dried under vacuum at 120-140° C. for about 3-6 hours to get crystalline mitapivat hemi sulfate Form VIII.

Another embodiment, the present disclosure is to provide crystalline mitapivat hemi sulfate Form IX, characterized by Powder X-ray diffraction pattern having 2θ angle positions at about 6.40, 12.97, 17.99, 19.70, 20.62 and 21.12±0.2° degrees two-theta.

In yet another embodiment, crystalline mitapivat hemi sulfate Form IX is further characterized by Powder X-ray diffraction pattern having 2θ angle positions at about 6.40, 12.12, 12.97, 13.81, 14.45, 15.01, 17.99, 19.19, 19.70, 20.13, 20.62, 21.12, 21.57, 22.35, 22.56, 23.00, 23.99, 24.48, 24.75, 25.35, 25.79, 26.27, 30.19 and 31.72±0.2° degrees two-theta.

Other embodiment of the present invention is to provide a process for the preparation of crystalline mitapivat hemi sulfate Form IX comprising the steps of:
a) suspending mitapivat in a mixture of isobutyl acetate and trifluoro ethanol;
b) adding sulfuric acid diluted with isobutyl acetate to the reaction mixture obtained in step (a);
c) heating the reaction mixture to 40-50° C.; and
d) isolating the crystalline mitapivat hemi sulfate Form IX.

Within the context of this embodiment, isolation can be done using any techniques in the art such as, decantation, filtration by gravity or suction, centrifugation. In particular useful embodiments the solid is isolated by filtration followed by suck-drying.

According to the present disclosure, mitapivat suspended in a mixture of isobutyl acetate and trifluoroethanol. To the reaction mixture added sulfuric acid solution diluted isobutyl acetate and is stirred for 20-30 minutes. The obtained precipitate is heated to 35-55° C. and stirred for 4-5 hours. The solid obtained is filtered and suck dried. The product obtained, which was identified as crystalline mitapivat hemi sulfate Form IX.

Another embodiment, the present disclosure is to provide crystalline mitapivat hemi sulfate Form X, characterized by Powder X-ray diffraction pattern having 2θ angle positions at about 7.21, 11.24, 12.19, 14.53 and 22.54±0.2° degrees two-theta.

In yet another embodiment, crystalline mitapivat hemi sulfate Form X is further characterized by Powder X-ray diffraction pattern having 2θ angle positions at about 7.21, 11.24, 12.19, 12.94, 13.23, 14.14, 14.53, 15.24, 16.01, 16.63, 17.11, 17.67, 18.14, 18.94, 19.54, 19.92, 20.78, 21.23, 22.54, 23.41, 23.74, 24.53, 24.91, 25.99, 26.87, 27.33, 28.34, 29.34, 30.23 and 30.71±0.2° degrees two-theta.

Other embodiment of the present invention is to provide a process for the preparation of crystalline mitapivat hemi sulfate Form X comprising the steps of:
a) suspending mitapivat in a mixture of isobutyl acetate and trifluoro ethanol;
b) adding sulfuric acid diluted with isobutyl acetate to the reaction mixture obtained in step (a);
c) heating the reaction mixture to 40-50° C.;
d) cooling the reaction mixture to 20-30° C.; and
e) isolating the crystalline mitapivat hemi sulfate Form X.

Within the context of this embodiment, isolation can be done using any techniques in the art such as, decantation, filtration by gravity or suction, centrifugation. In particular useful embodiments the solid is isolated by filtration followed by suck-drying.

According to the present disclosure, mitapivat suspended in a mixture of isobutyl acetate and trifluoroethanol. To the reaction mixture added sulfuric acid solution diluted isobutyl acetate and is stirred for 20-30 minutes. The obtained precipitate is heated to 35-55° C. and stirred for 2-3 hours. The reaction mixture then cooled to 20-27° C. and stirred for 14-17 hours. The solid obtained is filtered and suck dried. The product obtained, which was identified as crystalline mitapivat hemi sulfate Form X.

Another embodiment, the present disclosure is to provide crystalline mitapivat hemi sulfate Form XI, characterized by Powder X-ray diffraction pattern having 2θ angle positions at about 12.55, 13.93, 16.82, 24.36 and 29.11±0.2° degrees two-theta.

In yet another embodiment, crystalline mitapivat hemi sulfate Form XI is further characterized by Powder X-ray diffraction pattern having 2θ angle positions at about 12.55, 13.93, 16.32, 16.82, 17.52, 17.93, 19.23, 20.21, 21.08, 21.44, 22.92, 24.36, 25.76, 26.05 and 29.11±0.2° degrees two-theta.

In still another embodiment the present invention is to provide a process for the preparation of crystalline mitapivat hemi sulfate Form XI comprising drying crystalline mitapivat hemi sulfate Form IX or Form X or Form XII.

Within the context of this embodiment, drying is performed under atmospheric pressure or under reduced pressure.

According to the present disclosure, crystalline mitapivat hemi sulfate Form IX or crystalline mitapivat hemi sulfate Form X or crystalline hemi sulfate Form XII dried at 120-140° C. for about 3-6 hours to get crystalline mitapivat hemi sulfate Form XI.

Another embodiment, the present disclosure is to provide crystalline mitapivat hemi sulfate Form XII, characterized by Powder X-ray diffraction pattern having 2θ angle positions at about 6.58, 13.22, 15.18, 15.73, 19.82 and 20.75±0.2° degrees two-theta.

In yet another embodiment, crystalline mitapivat hemi sulfate Form XII is further characterized by Powder X-ray diffraction pattern having 2θ angle positions at about 6.02, 6.58, 9.57, 11.27, 12.25, 13.22, 14.52, 14.93, 15.18, 15.73, 17.21, 18.13, 19.21, 19.51, 19.82, 20.14, 20.75, 21.07, 21.62, 22.60, 23.15, 24.19, 24.84, 25.51, 25.85, 25.98 and 26.49±0.2° degrees two-theta.

In yet another embodiment, the physical stability of crystalline mitapivat hemi sulfate Form VII and crystalline mitapivat hemi sulfate Form XI were determined by storing the samples at 40° C./75% relative humidity (RH) and at 25° C./60% RH conditions for three months as shown in Table 1. The samples were tested by PXRD analysis. mitapivat hemi sulfate polymorphs, Form VII and Form XI, were found to be physically stable at 40° C./75% relative humidity (RH) and at 25° C./60% relative humidity (RH) conditions up to three months (refer Table 1).

TABLE 1
Summary of stability studies on Form VII and Form XI

| | | |
|---|---|---|
| | Mitapivat hemi | Mitapivat hemi |
| | sulfate Form VII | sulfate Form XI |
| Conditions | PXRD | PXRD |

at 40° C./75% RH

| | | |
|---|---|---|
| Initial | Form VII | Form XI |
| 1 month | Stable | Stable |
| 2 months | — | Stable |
| 3 months | Stable | Stable |

at 25° C./60% RH

| | | |
|---|---|---|
| 1 month | Stable | Stable |
| 2 months | Stable | Stable |
| 3 months | Stable | Stable |

In one embodiment, the present disclosure is to provide crystalline mitapivat Form I.

In another embodiment, the present disclosure is to provide crystalline mitapivat Form I, characterized by Powder X-ray diffraction pattern having 2θ angle positions at about 8.81, 11.22, 16.33, 22.24, 24.53 and 30.85±0.2° degrees two-theta.

In yet another embodiment, crystalline mitapivat Form I is further characterized by Powder X-ray diffraction pattern having 2θ angle positions at about 8.81, 10.84, 11.22, 16.33, 17.73, 21.07, 21.92, 22.23, 23.67, 23.92, 24.53 and 30.85±0.2° degrees two-theta.

In still another embodiment the present invention is to provide a process for the preparation of crystalline mitapivat Form I, comprising the steps of:
a) providing mitapivat in organic solvent;
b) cooling the reaction mixture to −5 to +5° C.; and
c) isolating crystalline mitapivat Form I.

Within the context of this embodiment, the organic solvent employed may include ethers, alcohols for example, but not limited to tetrahydrofuran, 2-methyl tetrahydrofuran, anisole, 1,4-dioxane, methyl tertiary butyl ether, diisopropyl ether, cyclopentyl methyl ether, 2-ethoxyethanol, 2-butoxyethanol, ethanol, methanol, n-propanol, n-butanol, 2-butanol, 2-propanol, 3-methyl-1-butanol, 1-pentanol, 2-methyl-1-propanol. In particular useful embodiments organic solvent is 2-ethoxyethanol.

Within the context of this embodiment, isolation can be done using any techniques in the art such as, decantation, filtration by gravity or suction, centrifugation. In particular useful embodiments the solid is isolated by filtration followed by suck-drying under vacuum.

According to the present disclosure, mitapivat is suspended in an organic solvent and stirred for about 16-45 hours. The reaction mass is cooled to −5 to +5° C., filtered and then suck-dried. The obtained solid is crystalline mitapivat Form I.

In another embodiment, the present disclosure is to provide crystalline mitapivat Form II.

Another embodiment, the present disclosure is to provide crystalline mitapivat Form II, characterized by Powder X-ray diffraction pattern having 2θ angle positions at about 4.87, 9.73, 17.80, 18.70, 24.42±0.2° degrees two-theta.

In yet another embodiment, crystalline mitapivat Form II is further characterized by Powder X-ray diffraction pattern having 2θ angle positions at about 4.87, 9.73, 12.15, 13.44, 14.23, 14.62, 15.59, 17.31, 17.80, 18.70, 19.54, 19.97, 20.84, 21.85, 22.06, 22.41, 22.90, 23.36, 23.80, 24.42, 25.53, 26.08, 26.67, 28.67 and 31.97±0.2° degrees two-theta.

In still another embodiment the present invention is to provide a process for the preparation of crystalline mitapivat Form II comprising the steps of:
a) dissolving mitapivat in an organic solvent at elevated temperature;
b) cooling the reaction mass to 20-30° C.;
c) adding an anti-solvent to the reaction mixture obtained in step (b); and
d) isolating crystalline mitapivat Form II.

Within the context of this embodiment, the organic solvent employed may include ethers, alcohols for example, but not limited to tetrahydrofuran, 2-methyl tetrahydrofuran, anisole, 1,4-dioxane, methyl tertiary butyl ether, diisopropyl ether, cyclopentyl methyl ether, 2-ethoxyethanol, 2-butoxyethanol, ethanol, methanol, n-propanol, n-butanol, 2-butanol, 2-propanol, 3-methyl-1-butanol, 1-pentanol, 2-methyl-1-propanol. In particular useful embodiments organic solvent is 2-ethoxyethanol.

Within the context of this embodiment, elevated temperature ranges from 50° C. to boiling point of solvent.

Within the context of this embodiment, the anti-solvent employed may include hydrocarbons, ethers, esters, for example, but not limited to heptane, n-hexane, hexanes, methylcyclohexane, pentane, toluene, diethyl ether, methyl tertiary butyl ether, diisopropyl ether, cyclopentyl methyl ether, anisole, ethyl acetate, n-propyl acetate, isopropyl acetate, n-butyl acetate, isobutyl acetate, methyl acetate and water. In particular useful embodiments anti-solvent is methyl tert-butyl ether.

Within the context of this embodiment, isolation can be done using any techniques in the art such as, decantation, filtration by gravity or suction, centrifugation. In particular useful embodiments the solid is isolated by filtration followed by suck-drying under vacuum.

According to the present disclosure, mitapivat is suspended in organic solvent and heated to elevated temperature of about 50° C. to boiling point of the solvent. The reaction mass is cooled to the temperature of about 20-30° C. and then added anti-solvent. The obtained solid is filtered and then suck-dried, which was identified as crystalline mitapivat Form II.

Another embodiment, the present disclosure is to provide crystalline mitapivat Form III, characterized by Powder X-ray diffraction pattern having 2θ angle positions at about 4.60, 1072, 13.46, 14.84, 18.71 and 22.33±0.2° degrees two-theta.

In yet another embodiment, crystalline mitapivat Form III is further characterized by Powder X-ray diffraction pattern having 2θ angle positions at about 4.60, 10.72, 11.44, 13.46, 14.19, 14.84, 16.06, 17.04, 17.46, 17.91, 18.71, 19.16, 20.92, 22.33, 22.77, 23.24 and 24.04±0.2° degrees two-theta.

Other embodiment of the present invention is to provide a process for the preparation of crystalline mitapivat Form III comprising the steps of:
a) dissolving mitapivat in acetic acid;
b) adding organic solvent and heating the reaction mixture; and
c) isolating crystalline mitapivat Form III.

Within the context of this embodiment, the organic solvent employed may include esters, ethers for example, but not limited to ethyl acetate, isopropyl acetate, isobutyl acetate, di ethyl ether, iso propyl ether, methyl tert-butyl ether (MTBE).

In particular useful embodiments organic solvent is methyl tert-butyl ether (MTBE).

Within the context of this embodiment, isolation can be done using any techniques in the art such as, decantation, filtration by gravity or suction, centrifugation. In particular useful embodiments the solid is isolated by filtration followed by suck-drying.

According to the present disclosure, mitapivat is dissolved in acetic acid and stirred for 20-30 minutes. To the reaction mixture added organic solvent, heated to 35-55° C. and is stirred for 20-30 minutes. The obtained precipitate is heated to 35-55° C. and stirred for 2-3 hours. The solid obtained is filtered and suck dried. The product obtained, which was identified as crystalline mitapivat Form III.

Another embodiment, the present disclosure is to provide crystalline mitapivat Form IV, characterized by Powder X-ray diffraction pattern having 2θ angle positions at about 6.70, 9.86, 13.37, 15.33, 15.52, 15.79, 16.14, 17.17, 19.04, 19.44, 19.68 and 23.33±0.2° degrees two-theta.

In yet another embodiment, crystalline mitapivat Form IV is further characterized by Powder X-ray diffraction pattern having 2θ angle positions at about 6.70, 7.68, 9.86, 13.37, 13.69, 15.33, 15.52, 15.79, 16.14, 17.17, 19.04, 19.44, 19.68, 21.46, 22.16, 22.46, 23.33, 24.05 and 25.77±0.2° degrees two-theta.

In still another embodiment the present invention is to provide a process for the preparation of crystalline mitapivat Form IV comprising drying crystalline mitapivat Form III.

According to the present disclosure, crystalline mitapivat hemi sulfate Form III dried under vacuum at 60-80° C. for about 6-8 hours to get crystalline mitapivat Form IV.

In still another embodiment the present invention is to provide a process for the preparation of crystalline mitapivat Form IV by treating crystalline mitapivat Form III in humidity.

Within the context of this embodiment, crystalline mitapivat Form III is exposed to relative humidity of 60-90% up to 40-55 hours. In particular useful embodiments 75% relative humidity is preferred.

In one embodiment, the present disclosure is to provide crystalline mitapivat mono sulfate Form I.

In another embodiment, the present disclosure is to provide crystalline mitapivat mono sulfate Form I, characterized by Powder X-ray diffraction pattern having 20 angle positions at about 4.49°, 13.54°, 15.87°, 21.320 and 22.680±0.2° degrees two-theta.

Another embodiment, crystalline mitapivat mono sulfate Form I is further characterized by Powder X-ray diffraction pattern having 2θ angle positions at about 13.15, 13.54, 15.87, 18.00, 19.78, 20.07, 20.47, 21.32, 22.03, 22.68, 23.06 and 24.08±0.2° degrees two-theta.

In yet another embodiment, crystalline mitapivat mono sulfate Form I is further characterized by Powder X-ray diffraction pattern having 2θ angle positions at about 4.49, 10.17, 11.66, 12.71, 13.15, 13.54, 14.68, 15.18, 15.87, 18.00, 19.78, 20.07, 20.47, 21.32, 22.03, 22.33, 22.68, 23.06, 23.53, 24.08, 25.57, 26.53, 30.18 and 30.34±0.2° degrees two-theta.

In still another embodiment the present invention is to provide a process for the preparation of crystalline mitapivat mono sulfate Form I, comprising the steps of:
a) dissolving mitapivat in acetic acid;
b) diluting sulfuric acid in acetic acid and adding to the reaction mixture obtained in step (a);
c) optionally seeding with crystalline mitapivat mono sulfate Form I; and
d) isolating crystalline mitapivat mono sulfate Form I.

Within the context of this embodiment 0.5 to 1.5 mole equivalents of sulfuric acid is added. In particular useful embodiments 1-1.2 mole equivalents of sulfuric acid is added.

Within the context of this embodiment, diluted sulfuric acid may be added to the reaction mixture in lot wise.

Within the context of this embodiment, isolation can be done using any techniques in the art such as, decantation, filtration by gravity or suction, centrifugation. In particular useful embodiments the solid is isolated by filtration followed by washing and suck-drying under vacuum.

According to the present disclosure, mitapivat is dissolved in acetic acid and added diluted sulfuric acid. The reaction mass is heated to 40-60° C. and stirred for about 15-20 hours. The reaction mass is cooled to 20 to 35° C., filtered and then suck-dried. The obtained solid is crystalline mitapivat mono sulfate Form I.

According to the present disclosure, mitapivat is dissolved in acetic acid and added one lot diluted sulfuric acid (˜0.5 equivalents). Next, the solution may be optionally seeded with crystalline mitapivat mono sulfate Form I. The reaction mass is heated to 40-60° C. and stirred for about 15-20 hours and then is cooled to 20 to 35° C. Added the second lot of diluted sulfuric acid (˜0.5 equivalents). The reaction mass is heated to 40-60° C. and stirred for about 15-20 hours, cooled to 20 to 35° C., filtered and then suck-dried. The obtained solid is crystalline mitapivat mono sulfate Form I.

In another embodiment, the present disclosure is to provide crystalline mitapivat mono sulfate Form II.

Another embodiment, the present disclosure is to provide crystalline mitapivat mono sulfate Form II, characterized by Powder X-ray diffraction pattern having 20 angle positions at about 15.05°, 17.64°, 20.12°, 21.730 and 24.070±0.2° degrees two-theta.

In another embodiment, crystalline mitapivat mono sulfate Form II is further characterized by Powder X-ray diffraction pattern having 2θ angle positions at about 10.67, 15.05, 17.64, 20.12, 21.73, 23.00 and 24.07±0.2° degrees two-theta.

In yet another embodiment, crystalline mitapivat mono sulfate Form II is further characterized by Powder X-ray diffraction pattern having 2θ angle positions at about 10.67, 13.36, 15.05, 15.29, 17.64, 19.02, 20.12, 20.29, 20.58, 21.23, 21.49, 21.73, 21.97, 22.47, 22.74, 23.00, 23.64, 24.07, 24.96 and 25.44±0.2° degrees two-theta.

In still another embodiment the present invention is to provide a process for the preparation of crystalline mitapivat mono sulfate Form II by treating crystalline mitapivat mono sulfate Form I in humidity.

Within the context of this embodiment, crystalline mitapivat mono sulfate Form I is exposed to relative humidity of 60-90%. In particular useful embodiments 90% relative humidity is preferred.

Another embodiment of the present invention is to provide a process for the preparation of crystalline mitapivat mono sulfate Form II comprising the steps of:
a) providing crystalline mitapivat mono sulfate Form I in a mixture of water and organic solvent; and
b) isolating crystalline mitapivat mono sulfate Form II.

Within the context of this embodiment, the organic solvent employed may include alcohols, esters, for example, but not limited to ethanol, propanol, isopropanol, 2-methyl-1-propanol, ethyl acetate, n-propyl acetate, isopropyl acetate, n-butyl acetate, isobutyl acetate, methyl acetate. In particular useful embodiments organic solvent is ethyl acetate.

Within the context of this embodiment, isolation can be done using any techniques in the art such as, decantation, filtration by gravity or suction, centrifugation. In particular useful embodiments the solid is isolated by filtration followed by washing and suck-drying under vacuum.

According to the present disclosure, mitapivat mono sulfate Form I is suspended in a mixture of water and an organic solvent. The reaction mass is stirred for about 15-20 hours, filtered and then suck-dried. The obtained solid is crystalline mitapivat mono sulfate Form II.

In another embodiment, the present disclosure is to provide crystalline mitapivat mono sulfate Form III.

Another embodiment, the present disclosure is to provide crystalline mitapivat mono sulfate Form III, characterized by Powder X-ray diffraction pattern having 2θ angle positions at about 15.33°, 20.51°, 20.78°, 21.07°, 22.190 and 24.990±0.2° degrees two-theta.

In another embodiment, crystalline mitapivat mono sulfate Form III is further characterized by Powder X-ray diffraction pattern having 2θ angle positions at about 12.20, 12.69, 13.16, 13.50, 15.33, 16.93, 17.80, 19.38, 19.73, 19.91, 20.51, 20.78, 21.07, 21.34, 22.00, 22.19, 23.07, 23.56, 24.49, 24.99 and 26.52±0.2° degrees two-theta.

In yet another embodiment, crystalline mitapivat mono sulfate Form III is further characterized by Powder X-ray diffraction pattern having 2θ angle positions at about 5.09, 10.21, 12.20, 12.69, 13.16, 13.50, 14.28, 15.33, 15.71, 16.93, 17.80, 19.38, 19.73, 19.91, 20.51, 20.78, 21.07, 21.34, 22.00, 22.19, 23.07, 23.56, 23.97, 24.49, 24.99, 26.52, 27.69, 28.15, 29.46, 30.52 and 30.93±0.2° degrees two-theta.

In still another embodiment the present invention is to provide a process for the preparation of crystalline mitapivat mono sulfate Form III by drying crystalline mitapivat mono sulfate Form I.

Within the context of this embodiment, drying is performed under atmospheric pressure or under reduced pressure. In particular useful embodiments drying is performed under reduced pressure.

In another embodiment, the present invention is to provide a process for the preparation of crystalline mitapivat mono sulfate Form III comprising the steps of:
a) providing crystalline mitapivat mono sulfate Form I in an organic solvent;
b) heating the reaction mixture; and
c) isolating crystalline mitapivat mono sulfate Form III.

Within the context of this embodiment, the organic solvent employed may include alcohols, esters, for example, but not limited to ethanol, propanol, isopropanol, 2-methyl-1-propanol, ethyl acetate, n-propyl acetate, isopropyl acetate, n-butyl acetate, isobutyl acetate, methyl acetate. In particular useful embodiments organic solvent is 2-methyl-1-propanol or isopropyl acetate.

Within the context of this embodiment, isolation can be done using any techniques in the art such as, decantation, filtration by gravity or suction, centrifugation. In particular useful embodiments the solid is isolated by filtration followed by suck-drying under vacuum.

According to the present disclosure, mitapivat mono sulfate Form I is suspended in an organic solvent. The reaction mixture was heated to about 50-70° C. and stirred. The reaction mass was filtered and then suck-dried, which was identified as crystalline mitapivat mono sulfate Form III.

Another embodiment, the present disclosure is to provide crystalline mitapivat mono sulfate Form IV, characterized by Powder X-ray diffraction pattern having 2θ angle positions at about 11.91°, 13.66°, 17.37°, 19.42°, 21.09°, 21.640 and 24.08°±0.2° degrees two-theta.

In yet another embodiment, crystalline mitapivat mono sulfate Form IV is further characterized by Powder X-ray diffraction pattern having 2θ angle positions at about 11.32, 11.91, 12.19, 13.66, 15.64, 15.87, 16.40, 16.99, 17.37, 17.62, 18.69, 18.90, 19.42, 19.69, 21.09, 21.64, 22.12, 22.45, 23.72, 24.08, 24.58, 25.53, 26.24, 26.43 and 28.41±0.2° degrees two-theta.

In yet another embodiment, crystalline mitapivat mono sulfate Form IV is further characterized by Powder X-ray diffraction pattern having 2θ angle positions at about 11.32, 11.91, 12.19, 12.89, 13.01, 13.29, 13.66, 15.64, 15.87, 16.40, 16.99, 17.37, 17.62, 17.91, 18.69, 18.90, 19.42, 19.69, 21.09, 21.64, 22.12, 22.45, 23.72, 24.08, 24.58, 25.01, 25.53, 26.02, 26.24, 26.43, 26.79, 27.51, 28.41, 28.67, 29.18, 30.08, 31.25 and 32.25±0.2° degrees two-theta.

In still another embodiment the present disclosure provides a process for the preparation of crystalline mitapivat mono sulfate Form IV by drying crystalline mitapivat mono sulfate Form II.

Within the context of this embodiment, drying is performed under atmospheric pressure or under reduced pressure. In particular useful embodiments drying is performed under reduced pressure.

Another embodiment of the present invention is to provide a process for the preparation of crystalline mitapivat mono sulfate Form IV comprising the steps of:
a) providing crystalline mitapivat mono sulfate Form II in an organic solvent;
b) heating the reaction mixture; and
c) isolating crystalline mitapivat mono sulfate Form IV.

Within the context of this embodiment, the organic solvent employed may include alcohols, hydrocarbons, ethers, for example, but not limited to ethanol, propanol, isopropanol, 2-methyl-1-propanol, 1-butanol, 2-butanol, heptane, n-hexane, methylcyclohexane, pentane, toluene, diethyl ether, methyl tertiary butyl ether, diisopropyl ether, cyclopentyl methyl ether, anisole. In particular useful embodiments organic solvent is 2-butanol, 1-butanol, heptane or anisole.

Within the context of this embodiment, isolation can be done using any techniques in the art such as, decantation, filtration by gravity or suction, centrifugation. In particular useful embodiments the solid is isolated by filtration followed by suck-drying under vacuum.

According to the present disclosure, mitapivat mono sulfate Form II is suspended in an organic solvent. The reaction mixture was heated to about 35-70° C. and stirred. The reaction mass was filtered and then suck-dried, which was identified as crystalline mitapivat mono sulfate Form IV.

Another embodiment, the present disclosure is to provide crystalline mitapivat mono sulfate Form V, characterized by Powder X-ray diffraction pattern having 2θ angle positions at about 12.04, 13.50, 17.15, 19.20, 20.71 and 23.18±0.2° degrees two-theta.

In yet another embodiment, crystalline mitapivat mono sulfate Form V is further characterized by Powder X-ray diffraction pattern having 2θ angle positions at about 9.87, 10.12, 11.22, 12.04, 13.50, 13.98, 14.63, 15.92, 17.15, 17.51, 19.20, 19.98, 20.71, 22.03, 22.77, 23.18, 23.52, 24.26, 25.13 and 26.33±0.2° degrees two-theta.

Other embodiment of the present invention is to provide a process for the preparation of crystalline mitapivat mono sulfate Form V comprising the steps of:
a) suspending mitapivat mono sulfate in a mixture of water and organic solvent;
b) heating and adding water; and
c) isolating crystalline mitapivat mono sulfate Form V.

Within the context of this embodiment, isolation can be done using any techniques in the art such as, decantation, filtration by gravity or suction, centrifugation. In particular useful embodiments the solid is isolated by filtration followed by suck-drying.

Within the context of this embodiment, nine moles of water is added in three equal lots.

Within the context of this embodiment, the organic solvent employed may include esters for example, but not limited to ethyl acetate, isopropyl acetate, isobutyl acetate. In particular useful embodiments organic solvent is ethyl acetate.

According to the present disclosure, mitapivat mono sulfate suspended in a mixture of water and organic solvent. Heated reaction mixture at 40-50° C. and stirred for 2-3 hours. Then added water to the reaction mass and stirred for 2-3 hours at 40-50° C. Again, water added to the reaction mass and stirred for 14-17 hours at 40-50° C. The solid obtained washed with water and suck-dried for 10-15 minutes. The solid obtained is further dried under vacuum at 40-50° C. for 14-17 hours to get mitapivat mono sulfate Form V.

Another embodiment, the present disclosure is to provide amorphous form of mitapivat mono sulfate.

Other embodiment of the present invention is to provide a process for the preparation of amorphous mitapivat mono sulfate comprising the steps of:
a) dissolving mitapivat in polar solvent;
b) adding sulfuric acid diluted water; and
c) lyophilizing to get amorphous mitapivat mono sulfate.

Within the context of this embodiment, the polar solvent employed may include water, acetone, acetonitrile, methanol, ethanol, ethyl acetate and mixtures thereof. In particular useful embodiments polar solvent is mixture of water and acetonitrile.

Within the context of this embodiment 0.5 to 1.5 mole equivalents of sulfuric acid is added. In particular useful embodiments 1-1.2 mole equivalents of sulfuric acid is added.

According to the present disclosure, mitapivat dissolved in polar solvent at elevated temperature of about 40° C. to boiling point of the solvent. To the reaction mixture added sulfuric acid solution diluted with water. Lyophilizing the reaction mixture for 14-17 hours to get amorphous mitapivat mono sulfate.

In one embodiment, the present disclosure is to provides a process for the preparation of mitapivat of formula I, which comprising the step of condensing the compound of formula III with 8-quinoline sulfonyl chloride of formula II.

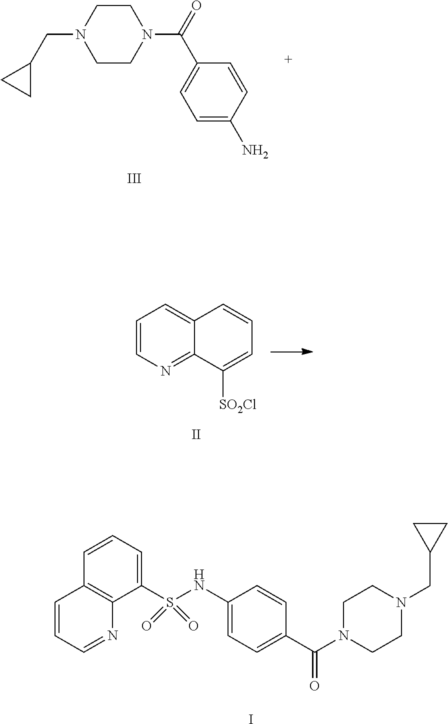

Within the context of this embodiment, the condensing step may be carried out in the presence of a base, which may be organic or inorganic base. The Inorganic base, for example selected from sodium hydroxide (NaOH) or potassium hydroxide (KOH). The organic base, for example selected from N-methylmorpholine (NMM), diisopropylethylamine, triethylamine, N,N′-dimethylpiperazine, N-methylpiperidine, pyridine, or mixtures thereof. In certain embodiments, the base used in this step is pyridine.

Within the context of this embodiment, the reaction is carried out in organic solvent, which may be aprotic solvent such as, for example, methylene dichloride (MDC), acetonitrile, tetrahydrofuran (THF), dimethylformamide (DMF), dimethyl acetamide (DMAc), ethyl acetate, toluene or mixtures thereof. In certain embodiments, the organic solvent used in this step is acetonitrile.

In another embodiment, the present disclosure is to provide a process for the preparation of compound of formula III,

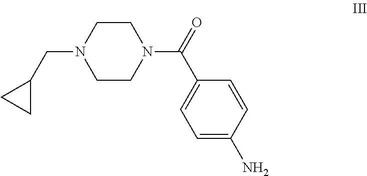 [SMILES: Nc1ccc(C(=O)N2CCN(CC3CC3)CC2)cc1]

which comprises:
   a) condensing cyclopropane piperazine of formula VI or its hydrochloride salt with 4-nitro benzoic acid of formula V to give the compound of formula IV; and

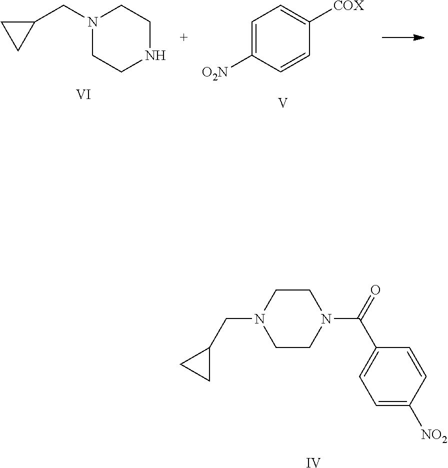

b) reducing the compound of formula IV to obtain the compound of formula III.

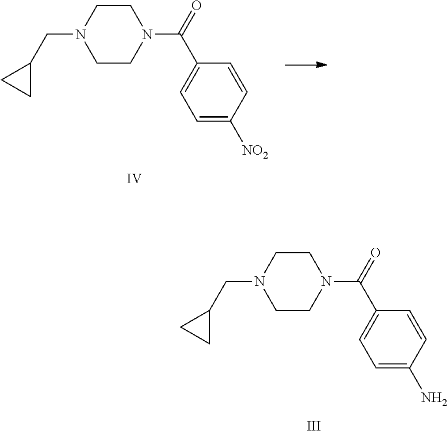

wherein X is selected from —OH or halogen (—Cl of —Br).

Within the context of this embodiment, the condensing step may be carried out (when X═OH) in the presence of a coupling agent, which may be carbonyldiimidazole (CDI), 1-Ethyl-3-(3′-dimethylaminopropyl)carbodiimide hydrochloride (EDC·HCl, or EDAC·HCl), N,N′-Dicyclohexylcarbodiimide (DCC) or mixtures thereof. In certain embodiments, the coupling agent used in this step is 1-ethyl-3-(3′-dimethylaminopropyl) carbodiimide hydrochloride (EDC·HCl, or EDAC·HCl). The condensing step may also be carried out in the presence of an additive such as, for example, hydroxyl benzotriazole (HOBt), 1-hydroxy-7-azabenzotriazole (HOAt), 6-chloro-1-hydroxy-1H-benzotriazole (Cl-HOBt), hydroxypyridines (HOPy), imidazole or its salts, 1,8-diazabicyclo[5.4.0]undec-7-en (DBU), dimethylaminopyridine (DMAP), or mixtures thereof. In certain embodiments, dimethylaminopyridine (DMAP) is used.

Within the context of this embodiment, the condensing step may also be carried out (when X═OH) in the presence of a chloroformate such as, for example methyl chloroformate, ethyl chloroformate or isobutyl chloroformate.

Within the context of this embodiment, the condensing step may also be carried out (when X=halogen) optionally in the presence of a base.

Within the context of this embodiment, the coupling reaction is carried out in organic solvent, which may be aprotic solvent such as, for example, methylene dichloride (MDC), acetonitrile, dioxane, tetrahydrofuran (THF), dimethylformamide (DMF), dimethyl acetamide (DMAc), ethyl acetate, toluene or mixtures thereof. In certain embodiments, the organic solvent used in this step is methylene dichloride (MDC).

Within the context of this embodiment, the reduction step may be carried out in the presence hydrogen and catalyst. The catalyst may be Palladium on carbon, Raney Nickel, Zinc-Acetic acid, Iron-HCl. In certain embodiments, the catalyst used in this step is Palladium on carbon.

Within the context of this embodiment, the reduction reaction is carried out in organic solvent, which may be methanol, ethanol, isopropanol, methylene dichloride (MDC), acetonitrile, tetrahydrofuran (THF), dimethylformamide (DMF), ethyl acetate or mixtures thereof. In certain embodiments, the organic solvent used in this step is methanol.

In another embodiment, the preset disclosure provides a process for the preparation of compound of formula III, which is shown in below scheme.

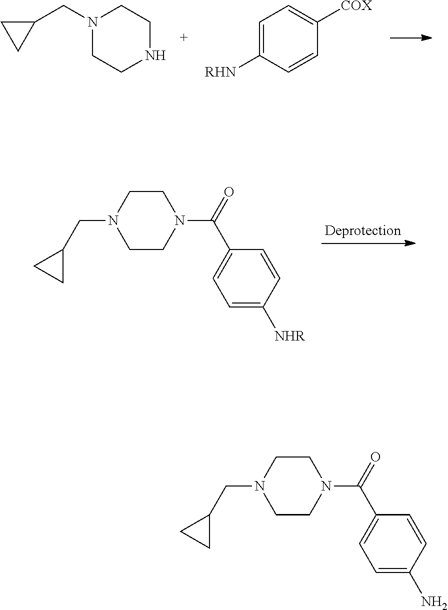

wherein X is selected from —OH or halogen (—Cl of —Br) and R is an amino protecting group.

In another embodiment, the preset disclosure provides a process for the preparation of mitapivat, or its pharmaceutically acceptable salts is shown in below scheme.

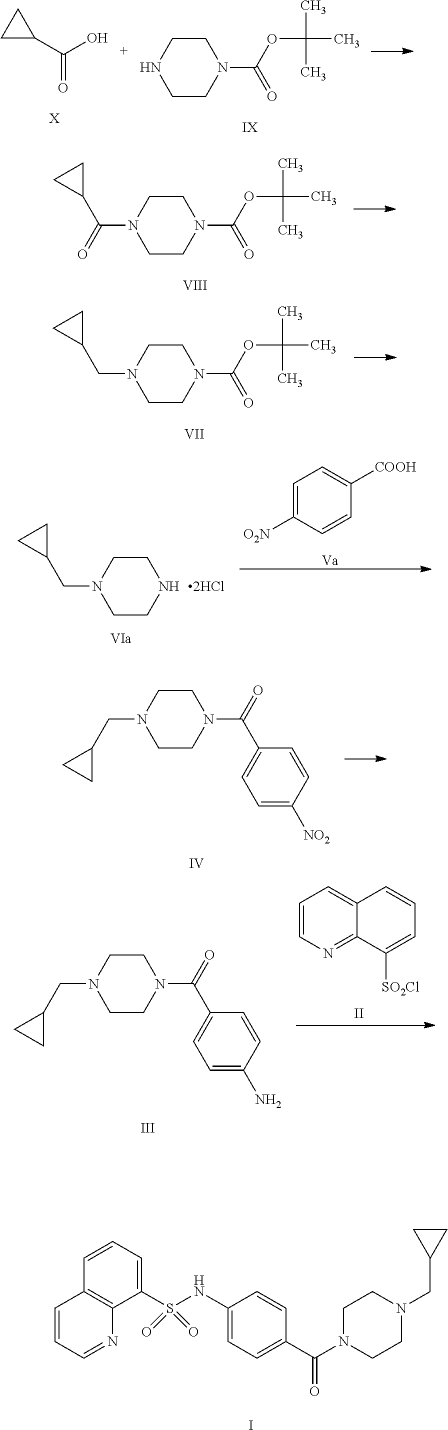

In another embodiment, the preset disclosure provides a process for the preparation of mitapivat, or its pharmaceutically acceptable salts is shown in below scheme.

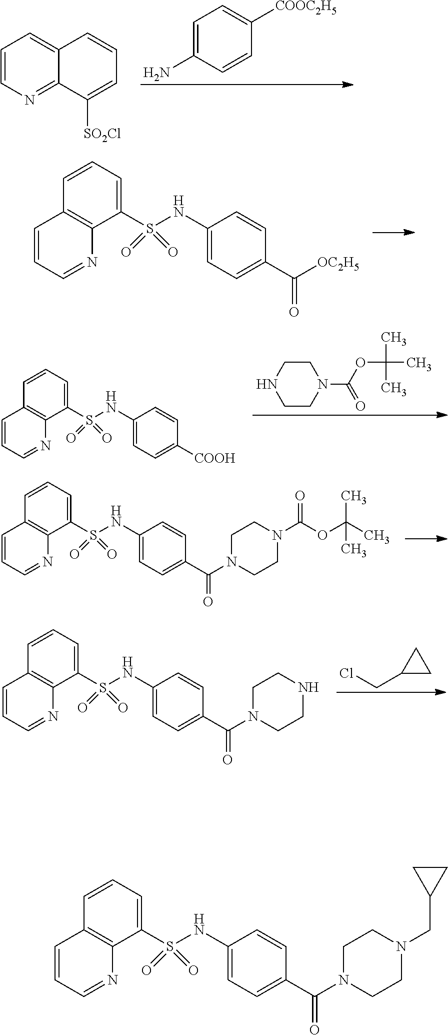

According to the present invention, the input mitapivat is prepared by any prior-art process for example PCT publication No. WO2011/002817.

In view of the above description and the examples below, one of ordinary skill in the art will be able to practice the invention as claimed without undue experimentation. The foregoing will be better understood with reference to the following examples that detail certain procedures for the preparation of molecules, compositions and Formulations according to the present invention. All references made to these examples are for the purposes of illustration. The following examples should not be considered exhaustive, but merely illustrative of only a few of the many aspects and embodiments contemplated by the present disclosure.

### EXAMPLES

### Example 1: Crystalline Mitapivat Hemi Sulfate Form I

Mitapivat (4 g) was suspended in a mixture of methanol (40 mL) and isobutyl acetate (76 mL). 0.52 equivalents of sulfuric acid diluted with isobutyl acetate (4 mL) was added at 25±5° C. The reaction mixture was then heated to 55±5° C. and maintained under stirring for 2 hours. A thin suspension was observed. The reaction volume was then reduced to ¼th volume under reduced pressure at 55±5° C. Methanol (40 mL) was then added to the reaction mixture and it was maintained at 55±5° C. for 2 hours. The reaction mixture was then cooled to 25±2° C. and stirred for 40 hours. Isobutyl acetate (40 mL) was added to the mixture, and it was maintained at 5±3° C. for 6 hours. The solid obtained was filtered, washed with chilled Isobutyl acetate (12 mL) and suck dried. The product obtained was identified by PXRD as crystalline Mitapivat hemi sulfate Form I.

Yield: 2.4 g

### Example 2: Crystalline Mitapivat Hemi Sulfate Form II

Mitapivat (2 g) was suspended in a mixture of methanol (20 mL) and isobutyl acetate (38 mL). 0.52 equivalents of sulfuric acid diluted with isobutyl acetate (2 mL) was added at 25±5° C. The resulting suspension was heated to 55±5° C. and maintained under stirring for 16 hours. A thin suspension was observed. The reaction volume was then reduced to ⅙th volume under reduced pressure at 55±5° C. The reaction mass was then cooled to 25±5° C. and stirred for 1 hour. The thick suspension was then further cooled to 5±3° C. and stirred for 6 hours. The solid obtained was filtered, washed with chilled isobutyl acetate (6 mL) and suck dried. The product obtained was identified by PXRD as crystalline Mitapivat hemi sulfate Form II.

Yield: 1.6 g

### Example 3: Crystalline Mitapivat Hemi Sulfate Form III

Mitapivat hemi sulfate Form II (1.6 g) obtained in Example 4 was dried under vacuum at 40° C. for 16 hours. The dried material was identified by PXRD as crystalline Mitapivat hemi sulfate Form III.

Yield: 1.5 g

### Example 4: Crystalline Mitapivat Hemi Sulfate Form III

Mitapivat (1 g) was suspended in a mixture of methanol (10 mL) and isobutyl acetate (19 mL). 0.52 equivalents of sulfuric acid diluted with isobutyl acetate (1 mL) was added at 25±5° C. The resulting suspension was heated to 55±5° C. and maintained under stirring for 4 hours. The reaction volume was then reduced to 1/6th volume under reduced pressure at 55±5° C. The reaction mixture was then slowly cooled to 25±5° C. and stirred for 14 hours. The thick suspension was then further cooled to 10±5° C. and stirred for 2 hour. The solid obtained was filtered, washed with chilled isobutyl acetate (3 mL), and dried at 50° C. under vacuum for 18 hours. The product obtained was identified by PXRD as crystalline Mitapivat hemi sulfate Form III.

Yield: 0.87 g

### Example 5: Crystalline Mitapivat Hemi Sulfate Form III

Mitapivat (4 g) was suspended in a mixture of methanol (40 mL) and isobutyl acetate (76 mL). 0.52 equivalents of sulfuric acid diluted with isobutyl acetate (4 mL) was added at 25±5° C. The resulting suspension was heated to 55±2° C. and maintained under stirring for 2 hours. The reaction volume was then reduced to 1/6th volume under reduced pressure at 55±5° C. The reaction mixture was then slowly cooled to 25±5° C. and stirred for 16 hours. The thick suspension was then further cooled to 5±3° C. and stirred for 6 hours. The solid obtained was filtered, washed with chilled isobutyl acetate (12 mL), and dried at 40° C. under vacuum for 16 hours. The product obtained was identified by PXRD as crystalline Mitapivat hemi sulfate Form III.

Yield: 3.7 g

### Example 6: Crystalline Mitapivat Hemi Sulfate Form IV

Mitapivat (500 mg) was dissolved in acetic acid (2 mL) at 60±5° C. The resulting clear solution was cooled to 25±5° C. and stirred for 15 minutes. Then added slowly 0.52 mole equivalents of sulfuric acid diluted with acetic acid (0.5 mL). The resulting clear solution was maintained under stirring for 16 hours at 25±5° C., then ethyl acetate (6 mL) was added at the same temperature. The resulting hazy solution was stirred for 2 hours at 25±5° C. The precipitated solid was then filtered and suck-dried for 1 hour. The product obtained was identified by PXRD as novel crystalline Mitapivat hemi sulfate Form IV.

Yield: 445 mg

### Example 7: Crystalline Mitapivat Hemi Sulfate Form IV

Mitapivat (5 g) was dissolved in acetic acid (20 mL) at 65±5° C. The resulting clear solution was cooled to 25±5° C. and filtered through hyflo to remove any undissolved particulate matter. To the clear solution then added slowly 0.52 mole equivalents of sulfuric acid diluted with acetic acid (5 mL). The resulting clear solution was maintained under stirring at 25±5° C. for 1 hour, then slowly added ethyl acetate (100 mL). The resulting hazy solution was stirred at 25±5° C. for 1 hour. The resulting solid was then filtered, washed with ethyl acetate (5 mL), and dried under vacuum at 40° C. for 15 hours. The product obtained was identified by PXRD as novel crystalline Mitapivat hemi sulfate Form IV.

Yield: 5.6 g

### Example 8: Crystalline Mitapivat Hemi Sulfate Form IV

Mitapivat hemi sulfate (6.8 g) was dissolved in acetic acid (34 mL) at 75±5° C. The resulting solution was then filtered through hyflo to remove any dissolved particulate matter. The particle-free solution was then cooled to 25±5° C. and then slowly added ethyl acetate (170 mL) under stirring. The resulting hazy solution was stirred for 1 hour at 25±5° C. The resulting solid was filtered, washed with ethyl acetate (3 mL) and suck-dried for 10-15 min. The solid obtained was identified by PXRD as novel crystalline Mitapivat hemi sulfate Form IV.

Yield: 6.9 g

### Example 9: Crystalline Mitapivat Hemi Sulfate Form V

In a RBF, charged mitapivat sulfate Form IV (1 g), ethyl acetate (10 mL) and water (32 mg) at 25±5° C. Heated the contents to 60-65° C. and the resulting suspension was maintained under stirring for 2-3 hours at 60-65° C. The reaction mass was then filtered at 60-65° C. and suck-dried for 10-15 minutes at 25-30° C. The solid obtained was further dried under vacuum at 100° C. for 8-10 hours. The product obtained was identified by PXRD as novel crystalline Mitapivat hemi sulfate Form V.

Yield: 0.8 g

### Example 10: Crystalline Mitapivat Hemi Sulfate Form VI

Suspend Mitapivat hemi sulfate (100 mg) in trifluoroethanol (0.2 mL) at 25±5° C. and heated the contents to 75±2° C. The resulting clear solution was cooled to 25±2° C. and added water (0.5 mL). The obtained hazy solution was stirred for 3 days at 25±2° C. The resulting solid was filtered, washed with water (0.2 mL) and suction dried for 30 minutes. The product obtained was identified by PXRD as novel crystalline Mitapivat hemi sulfate Form VI.

Yield: 95 mg.

### Example 11: Crystalline Mitapivat Hemi Sulfate Form VI

Dissolve Mitapivat freebase (0.5 g) in trifluoroethanol (1 mL) at 25±5° C. The clear solution was filter through 0.45p filter and then washed with trifluoroethanol (1 mL). In another RBF, charge water (7.5 mL), sulfuric acid (58 mg) and cool the reaction mass to 5±3° C. To the clear solution then added seeds of Mitapivat hemi sulfate Form VI (5 mg). To this hazy solution added the previously prepared freebase solution at 5±3° C. and maintained under stirring for 6 hours at 0-5° C. The reaction mass temperature was then raised to 20-25° C. and stirred for 8 hours. The reaction mass was then cooled to 0-5° C. and stirred for 1 hour. The solid obtained was filtered and suck-dried for 30 minutes. The product obtained was identified by PXRD as novel crystalline Mitapivat hemi sulfate Form VI.

Yield: 0.56 g

### Example 12: Crystalline Mitapivat Hemi Sulfate Form VII

Mitapivat hemi sulfate Form IV (200 mg) obtained as per the example above was dissolved in trifluoroethanol (1 mL) at 80±5° C. The solution was slowly evaporated on a laboratory rota evaporator at 70±2° C. till an oily residue. Cool the reaction mass to 25±5° C. and then added DM water (1 mL) to the flask and the resulting clear solution was sonicated for 5 minutes at 25±5° C. The material obtained was filtered and suck-dried. The product obtained was identified by PXRD as novel crystalline Mitapivat hemi sulfate Form VII.

Yield: 188 mg

### Example 13: Crystalline Mitapivat Hemi Sulfate Form VII

Mitapivat hemi sulfate (500 mg) was dissolved in trifluoroethanol (2 mL) at 75±5° C. The resulting clear solution was cooled to 25±5° C. In a separate round-bottom flask DM water (5 mL) was taken, cooled to 5±3° C., then added seeds of Mitapivat hemi sulfate Form VII (5 mg) and maintained under stirring. To this hazy solution previously prepared Mitapivat hemi sulfate solution in trifluoroethanol was slowly added. The milky white suspension obtained was then maintained under stirring at 5±3° C. for 1 hour. The resulting solid was filtered, and suction dried for 30 minutes. The product obtained was identified by PXRD as novel crystalline Mitapivat hemi sulfate Form VII.

Yield: 400 mg

### Example 14: Crystalline Mitapivat Hemi Sulfate Form VII

In a RBF, charged Mitapivat (500 mg) and trifluoroethanol (2 mL) at 25±5° C. Heated the contents to 75±5° C. and stirred for 5-10 min at 75±5° C. The obtained clear solution was then cooled to 25±5° C. In another RBF, charged water (2.5 mL) and added sulfuric acid solution prepared by dissolving 58 mg sulfuric acid in 0.5 mL water. Cooled the reaction mass to 5±3° C. and then added previously prepared Mitapivat solution in trifluoroethanol slowly at 5±3° C. To the obtained hazy solution added water (1.5 mL) and maintained under stirring for 1 h at 5±3° C. The obtained solid was filtered at 2-8° C., suck-dried for 30 minutes at 25±2° C. and dried under vacuum at 40° C. for 72 hours. The product obtained was identified by PXRD as novel crystalline Mitapivat hemi sulfate Form VII.

Yield: 420 mg

### Example 15: Crystalline Mitapivat Hemi Sulfate Form VII

In RBF, charged Mitapivat base (2 g) and trifluoroethanol (4 mL) at 28±2° C. and stirred for 5-10 minutes. The resulting clear solution was filtered through 0.45p filter and washed with trifluoroethanol (4 mL) to remove any dissolved particulate matter. To the obtained clear solution added water (4 mL) and further cooled to 0-5° C. Then added Mitapivat hemi sulfate Form VII (20 mg) and followed by addition of sulfuric acid (232 mg, 0.52 eq) diluted in water (18 mL) at 0-5° C. for 5-10 minutes. The obtained hazy solution was stirred for 5 hours at 0-5° C., then raised the reaction mass temperature to 25-30° C. and stirred for 14 hours. The reaction mass was then cooled to 0-5° C. and stirred for 9 hours, then raised the reaction mass temperature to 25-30° C. and stirred for 14 hours. The reaction mass was then cooled to 0-5° C. and stir for 2 hours. The solid obtained was filtered, washed with mixture of trifluoroethanol (0.8 mL) and water (2.2 mL) and suck-dried for 30 minutes. and dried under vacuum at 40° C. for 18 hours. The product obtained was identified by PXRD as novel crystalline Mitapivat hemi sulfate Form VII.

Yield: 1.9 g

### Example 16: Crystalline Mitapivat Hemi Sulfate Form VII

In RBF, charged Mitapivat freebase (0.5 g) and trifluoroethanol (1 mL) at 25±5° C. The resulting clear solution was filtered through 0.45p filter and washed with trifluoroethanol (1 mL) to remove any dissolved particulate matter. To the obtained clear solution added water (1 mL) and further cooled to 0-5° C. Then added solution of sulfuric acid (58 mg) in water (3.5 mL) and followed by addition of seeds of Mitapivat hemi sulfate Form VII (5 mg). The obtained hazy solution was stirred for 6 h at 0-5° C., then raised the reaction mass temperature to 20-25° C. and stirred for 8 hours. The reaction mass was then cooled to 0-5° C. and stirred for 1 hour. The solid obtained was filtered, washed with water (0.5 mL) and suck-dried for 30 minutes. The product obtained was identified by PXRD as novel crystalline Mitapivat hemi sulfate Form VII.

Yield: 0.48 g

### Example 17: Crystalline Mitapivat Hemi Sulfate Form VII

In RBF, charged Mitapivat base (2 g) and trifluoroethanol (4 mL) at 28±2° C. and stirred for 5-10 minutes. The resulting clear solution was filtered through 0.45p filter and washed with trifluoroethanol (4 mL) to remove any dissolved particulate matter. To the obtained clear solution added water (4 mL) and further cooled to 0-5° C. Then added seeds of Mitapivat hemi sulfate Form VII (20 mg) followed by addition of Conc. sulfuric acid (232 mg, 0.52 mole eq.) diluted in water (18 mL) at 0-5° C. for 5-10 minutes. The obtained hazy solution was stirred for 24 h at 0-5° C. The solid obtained was filtered, washed with mixture of trifluoroethanol (0.8 mL) and water (2.2 mL) and suck-dried for 30 minutes. and dried at 40° C. under vacuum for 16 h. The product obtained was identified by PXRD as novel crystalline Mitapivat hemi sulfate Form VII.

Yield: 1.8 g

### Example 18: Crystalline Mitapivat Hemi Sulfate Form VIII

Mitapivat hemi sulfate Form VI & VII obtained as per above examples was placed in a petri dish and subjected to drying under vacuum at 130° C. for 3-6 hours. The resulting solid was identified by PXRD as novel crystalline Mitapivat hemi sulfate Form VIII.

### Example 19: Crystalline Mitapivat Hemi Sulfate Form IX

In RBF, charged Mitapivat freebase (1.0 g), Isobutyl acetate (29 mL) and trifluoroethanol (10 mL) at 25±5° C. Stir the reaction mass for 5-10 minutes. To the resulting hazy solution added solution of sulfuric acid (113 mg) in isobutyl acetate (1 mL) at 25±5° C. and stirred for 20-30 minutes. The obtained precipitate was heated to 45±2° C. and stirred for 5 hours. The resulting solid was filtered and suction-dried for 30 minutes. The product obtained was identified by PXRD as novel crystalline Mitapivat hemi sulfate Form IX.

Yield: 0.5 g

### Example 20: Crystalline Mitapivat Hemi Sulfate Form X

In RBF, charged Mitapivat freebase (0.5 g), Isobutyl acetate (14.5 mL) and trifluoroethanol (5 mL) at 25±5° C. Stir the reaction mass for 10-15 minutes. To the resulting hazy solution added solution of sulfuric acid (58 mg) in isobutyl acetate (0.5 mL) at 25±5° C. and stirred for 20-30 minutes. The obtained precipitate was heated to 45±2° C. and stirred for 3 hours. Cooled the reaction mass to 25±2° C. and stirred for 16 hours at 25±2° C. The resulting solid was filtered, and suction-dried for 30 minutes. The product obtained was identified by PXRD as novel crystalline Mitapivat hemi sulfate Form X.

Yield: 0.6 g

### Example 21: Crystalline Mitapivat Hemi Sulfate Form XI

Mitapivat hemi sulfate Form IX & X obtained as per above examples was placed in a petri dish and subjected to drying under vacuum at 130° C. for 3-6 hours. The resulting solid was identified by PXRD as novel crystalline Mitapivat hemi sulfate Form XI.

### Example 22: Crystalline Mitapivat Hemi Sulfate Form XI

In RBF, charged Mitapivat base (50 g), isobutyl acetate (550 mL) and trifluoroethanol (325 mL) at 27±2° C. To the reaction mass add slowly Conc. H₂SO₄ (0.52 mole eq.) diluted with 37.5 mL isobutyl acetate and maintained under stirring at 27±2° C. for 30 minutes which resulting clear solution. Then raise the temperature to 45±2° C. and maintained under stirring for 5 hours. Precipitation was observed. The resulting reaction mass was cooled to 28±2° C., maintained for 16 hours at 28±2° C., filtered, washed with isobutyl acetate (100 mL) and suck-dried for 45 min. The wet material was dried at 100° C. under vacuum for 16 h. The solid obtained was identified by PXRD as Mitapivat hemi sulfate Form XI.

Yield: 51 g

### Example 23: Crystalline Mitapivat Hemi Sulfate Form XI

In RBF, charged mitapivat base (1 g), isopropyl acetate (11 mL) and trifluoroethanol (6.5 mL) at 27±2° C. To the reaction mass add slowly Conc. H₂SO₄ (0.52 mole eq.) diluted with 0.6 mL isopropyl acetate and maintained under stirring at 28±2° C. for 30 minutes resulting clear solution. Then raise the temperature to 45±2° C. and maintained under stirring for 5 hours. Precipitation was observed. The resulting reaction mass was cooled to 28±2° C., maintained for 16 hours at 28±2° C. filtered the reaction mass, washed with isopropyl acetate (2 mL) and suck-dried for 30 minutes. Wet material was dried at 100° C. under vacuum for 16 hours. The solid obtained was identified by PXRD as Mitapivat hemi sulfate Form XI.

Yield: 0.8 g

### Example 24: Crystalline Mitapivat Hemi Sulfate Form XI

In RBF charged, mitapivat base Form III (1 g) and trifluoroethanol (2 mL) at 28±2° C. Stir the reaction mass for 15-20 minutes. to get clear solution. The reaction mass was then distilled under vacuum using rotary evaporator at 70±2° C. Then added trifluoroethanol (2 mL) and isobutyl acetate (3 mL) at 70±2° C., the clear solution was distilled under vacuum at 70±2° C. Then added isobutyl acetate (8.3 mL) and trifluoroethanol (4.9 mL) at 25±2° C. and the suspension was stirred for 10 minutes at 25±2° C. Added slowly Conc. H₂SO₄ (85 mg, 0.52 mole eq.) diluted with 1.0 mL isobutyl acetate and maintained under stirring at 25±2° C. for 15 minutes resulting clear solution. Then raise the temperature to 45±2° C. and maintained under stirring for 4 hours. Precipitation was observed. The resulting reaction mass was cooled to 25±2° C. and maintained for 34 hours. Filtered the reaction mass, washed with isopropyl acetate (2 mL) and suck-dried for 30 minutes. The wet material was dried at 100° C. under vacuum for 14 hours. The solid obtained was identified by PXRD as Mitapivat hemi sulfate Form XI.

Yield: 0.35 g

### Example 25: Crystalline Mitapivat Hemi Sulfate Form XI

In RBF charged, mitapivat base (1 g), Ethyl acetate (11 mL) and trifluoroethanol (6.5 mL) at 28±2° C. To the reaction mass add slowly Conc. H₂SO₄ (0.52 mole eq.) diluted with 0.6 mL ethyl acetate and maintained under stirring at 28±2° C. for 30 minutes resulting clear solution. Then raise the temperature to 45±2° C. and maintained under stirring for 5 hours. Precipitation was observed. The resulting reaction mass was cooled to 28±2° C., stirred for 16 hours, filtered, washed with Ethyl acetate (2 mL) and suck-dried for 30 minutes. The wet material was dried at 100° C. under vacuum for 16 hours. The solid obtained was identified by PXRD as Mitapivat hemi sulfate Form XI.

Yield: 0.88 g

### Example 26: Crystalline Mitapivat Hemi Sulfate Form XI

In RBF charged, mitapivat base (1 g), n-Propyl acetate (11 mL) and trifluoroethanol (6.5 mL) at 28±2° C. To the reaction mass add slowly Conc. H₂SO₄ (0.52 mole eq.) diluted with 0.6 mL n-Propyl acetate and maintained under stirring at 28±2° C. for 30 minutes resulting clear solution. Then raise the temperature to 45±2° C. and maintained under stirring for 5 hours. Precipitation was observed. The resulting reaction mass was cooled to 28±2° C., stirred for 16 hours, filtered, washed with n-Propyl acetate (2 mL) and suck-dried for 30 minutes. The wet material was dried at 100° C. under vacuum for 16 hours. The solid obtained was identified by PXRD as Mitapivat hemi sulfate Form XI.

Yield: 0.86 g

### Example 27: Crystalline Mitapivat Hemi Sulfate Form XI

Charged Mitapivat hemi sulfate Form XII (2 g) into a petri-dish and dried at 100° C. under vacuum for 4 hours. The resulting solid was identified by PXRD as Mitapivat hemi sulfate Form XI.

### Example 28: Crystalline Mitapivat Hemi Sulfate Form XII

In RBF charged, mitapivat base (2 g), isobutyl acetate (22 mL) and trifluoroethanol (13 mL) at 25±2° C. To the reaction mass add slowly Conc. H₂SO₄ (0.52 mole eq.) diluted with 1.5 mL isobutyl acetate and maintained under stirring at 25±2° C. for 30 minutes resulting clear solution. Then raise the temperature to 45±2° C. and maintained under stirring for 5 hours. Precipitation was observed. The resulting reaction mass was cooled to 25±2° C., stirred for 16 hours at 25±2° C., filtered, washed with isobutyl acetate (4 mL) and suck-dried for 30 minutes. The wet material obtained was identified by PXRD as Mitapivat hemi sulfate Form XII.

Yield: 2.5 g

### Example 29: Crystalline Mitapivat Form I

Mitapivat (1 g) was suspended in 2-ethoxyethanol (80 mL) at 25±2° C. The suspension was stirred at 25±2° C. for 40 hours. Then the reaction mixture was cooled to 0±2° C., stirred for 1 hour, filtered and suck dried. The solid obtained was identified by PXRD as crystalline mitapivat freebase Form I.

Yield: 0.2 g

### Example 30: Crystalline Mitapivat Form II

Mitapivat (2 g) was suspended in 2-ethoxyethanol (20 mL) at 25±2° C. and heated to 80° C. to obtain a clear solution. The solution was then slowly cooled to 50±2° C., a hazy suspension was observed. The reaction mass was further cooled to 25±2° C. and maintained under stirring for 16 hours. A thin hazy suspension was observed. Methyl tert-butyl ether (50 mL) was slowly added to the resulting hazy solution and stirring was continued at 25±2° C. for 3 hours. The solid obtained was filtered and suction dried. The solid obtained was identified by PXRD as crystalline Mitapivat freebase Form II.

Yield: 1.8 g

### Example 31: Crystalline Mitapivat Form III

Mitapivat (500 mg) was dissolved in acetic acid (2 mL) at 25±5° C. The clear solution obtained was stirred at 25±5° C. for 20 minutes and methyl tert-butyl ether (8 mL) was added at the same temperature. The resulting hazy solution was heated to 40±5° C. and stirred at that temperature for 20 minutes. The resulting solid was filtered and suck-dried for 20 minutes. The product obtained was identified by PXRD as novel crystalline Mitapivat Form III.

Yield: 560 mg

### Example 32: Crystalline Mitapivat Form IV

Mitapivat base Form III (0.2 g) was charged into petri-dish and dried under vacuum at 70° C. for 7 hours. The resulting solid was identified by PXRD as crystalline mitapivat Form IV.

### Example 33: Crystalline Mitapivat Form IV

Mitapivat base Form III (0.2 g) was charged into petri-dish and expose to 75% relative humidity condition up to 48 hours. The resulting solid was identified by PXRD as crystalline mitapivat Form IV.

### Example 34: Crystalline Amorphous Mitapivat Mono Sulfate

Mitapivat (500 mg) was dissolved in of acetonitrile-water mixture (2:1 v/v) (15 mL) at 50±5° C. and stirred for 15 minutes. To the clear solution was added one mole equivalent of sulfuric acid diluted in water (1 mL). The resulting clear solution was made particle-free by filtration, then freezed using a mixture of dry-ice and methanol and subjected to lyophilization using Labocon lyophilizer (Model: LFD-BT-104) for 16 hours. The solid obtained was identified by PXRD as the amorphous form of Mitapivat mono sulfate.

Yield: 500 mg

### Example 35: Crystalline Mitapivat Mono Sulfate Form I

Mitapivat (200 mg) was dissolved in acetic acid (2 mL) at 25±5° C. To the clear solution slowly added 0.52 mole equivalents of sulfuric acid diluted with 0.2 mL acetic acid. The resulting clear solution was slowly heated to 50±2° C. and stirred at that temperature for 16 hours. The reaction mass was then cooled to 25±5° C. and stirred at that temperature for 2 hours. The resulting solid was filtered, washed with 0.5 mL acetic acid and suck dried. The product obtained was identified by PXRD as crystalline Mitapivat mono sulfate Form I.

### Example 36: Crystalline Mitapivat Mono Sulfate Form I

Mitapivat (2 g) was suspended in 20 mL acetic acid at 25±5° C. The suspension was stirred at the same temperature for 15-20 minutes to obtain a clear solution. Then added 0.52 equivalents of sulfuric acid diluted with 2 mL acetic acid and added Form I seeds (10 mg). The reaction mass was then heated to 50±2° C. and maintained under stirring for 2 hours, then cooled to 25±5° C. and added second lot of sulfuric acid (0.52 equivalents) diluted with 2 mL acetic acid. The reaction mass was then heated to 50±5° C. and maintained under stirring at 50±5° C. for 16 hours. The resulting suspension was cooled to 25±5° C. and stirred for 2 hours. The solid obtained was filtered, washed with 1 mL acetic acid and suck dried. The product obtained was identified by PXRD as crystalline Mitapivat mono sulfate Form I.

Yield: 1.6 g

### Example 37: Crystalline Mitapivat Mono Sulfate Form I

Mitapivat (1 g) was suspended in 4 mL acetic acid and heated to 65±5° C. to obtain a clear solution. The resulting clear solution was cooled to 25±5° C., added 0.52 equivalents of sulfuric acid diluted with 1 mL acetic acid. The solution was then heated to 50-5° C., added Form I seeds (5 mg) and maintained under stirring at 50±5° C. for 16 hours. Precipitate was not observed, and seeds were dissolved. To the resulting solution second lot of sulfuric acid (0.52 equivalents) diluted with 0.5 mL acetic acid was added. Hazy solution was observed. The reaction mass was then maintained under stirring at 50±5° C. for 2 hours. White solid material precipitated. The resulting suspension was then cooled to 25±5° C. and stirred for 1 hour. The solid obtained was filtered, washed with 1 mL acetic acid and suck dried. The product obtained was identified by PXRD as crystalline Mitapivat mono sulfate Form I.

Yield: 0.9 g

### Example 38: Crystalline Mitapivat Mono Sulfate Form II

Mitapivat mono sulfate Form I (800 mg) obtained as above was placed in an open petri-dish and exposed to 90% relative humidity for 16 hours. The resulting solid was identified by PXRD as Mitapivat mono sulfate Form II.

Yield: 760 mg

### Example 39: Crystalline Mitapivat Mono Sulfate Form II

Mitapivat mono sulfate Form I (100 mg) was suspended in 0.7 mL ethyl acetate at 25±5° C. and then charged DM water in varying quantities (ref. Table 1). The resulting suspensions were maintained under stirring at 25±5° C. for 15 hours, then filtered, washed with 0.3 mL ethyl acetate in each case and suck-dried. The solid obtained in each case was confirmed by PXRD as Mitapivat mono sulfate Form II.

Yield: 85 mg

TABLE 1

| | | Ethyl | | |
|---|---|---|---|---|
| Input | Quantity | acetate | Water | |
| polymorph | (mg) | (mL) | (mg) | PXRD |
| Form I | 100 | 0.7 | 5 | Form II |
| Form I | 100 | 0.7 | 6.5 | Form II |
| Form I | 100 | 0.7 | 10 | Form II |

### Example 40: Crystalline Mitapivat Mono Sulfate Form III

Mitapivat mono sulfate Form I (200 mg) obtained as above was dried at 160° C. under vacuum for 1 hour. The resulting solid was identified by PXRD as Mitapivat mono sulfate Form III.

Yield: 180 mg

### Example 41: Crystalline Mitapivat Mono Sulfate Form III

Mitapivat mono sulfate Form I (1.5 g) obtained as above was dried at 130° C. under vacuum for 7 hours. The resulting solid was identified by PXRD as Mitapivat mono sulfate Form III.

Yield: 1.35 g

### Example 42: Crystalline Mitapivat Mono Sulfate Form III

Mitapivat mono sulfate Form I (500 mg) obtained as above was suspended in different solvents (refer Table 2) at 25±5° C. The suspension was then heated to 60±5° C., stirred at that temperature for 2 h, filtered and suck dried. The solid isolated was identified by PXRD as Mitapivat mono sulfate Form III.

Yield: 400 mg

TABLE 2

| Input | | | | |
|---|---|---|---|---|
| Polymorph | Quantity | Solvent | Volume | PXRD |
| Form I | 500 mg | 2-Methyl-1-Propanol | 10 mL | Form III |
| Form I | 500 mg | Isopropyl acetate | 25 mL | Form III |

### Example 43: Crystalline Mitapivat Mono Sulfate Form IV

Mitapivat mono sulfate Form II (1.2 g) obtained as above was dried at 100° C. under vacuum for 7 hours. The resulting solid was identified by PXRD as Mitapivat mono sulfate Form IV.

Yield: 1.1 g

### Example 44: Crystalline Mitapivat Mono Sulfate Form IV

Mitapivat mono sulfate Form II (500 mg) was suspended in 2-butanol at 25±5° C. The suspension was heated up to 60±5° C., stirred at that temperature for 2 hours, filtered and suck dried. The solid isolated was identified by PXRD as Mitapivat mono sulfate Form IV.

Yield: 430 mg

### Example 45: Crystalline Mitapivat Mono Sulfate Form IV

Mitapivat mono sulfate Form II obtained as above was suspended in different solvents (refer Table 3) at 25±5° C. The suspension was then heated to 60±5° C. or 40±5° C. and stirred at that temperature for 2 hours. The resulting reaction mixture was filtered and suck dried. The solid isolated was identified by PXRD as Mitapivat mono sulfate Form IV.

TABLE 3

| Input | Quantity | | Volume | | Temperature | |
|---|---|---|---|---|---|---|
| Polymorph | (mg) | Solvent | (mL) | | (° C.) | PXRD |
| Form II | 500 | 2-Butanol | 10 | mL | 60 ± 5 | Form IV |
| Form II | 100 | Heptane | 5 | mL | 40 ± 5 | Form IV |
| Form II | 100 | Anisole | 5 | mL | 40 ± 5 | Form IV |
| Form II | 100 | 1-Butanol | 5 | mL | 40 ± 5 | Form IV |

### Example 46: Crystalline Mitapivat Mono Sulfate Form V

In a RBF, charged Mitapivat mono sulfate Form I (0.5 g), ethyl acetate (5 mL) and water (44 mg) at 25±5° C. Heated the contents to 40-45° C. and the resulting suspension was maintained under stirring for 2-3 hours at 40-45° C. Then added water (44 mg) to the reaction mass and maintained under stirring for 2-3 hours at 40-45° C. Again, added water (44 mg) to the reaction mass and stirred for 16 hours at 40-45° C. The solid obtained was filtered, washed with water (0.5 mL) and suck-dried for 10-15 minutes. The solid obtained was further dried under vacuum at 40° C. for 16 hours. The product obtained was identified by PXRD as novel crystalline Mitapivat mono sulfate Form V.

Yield: 0.40 g

### Example 47: Preparation of Compound of Formula VIII

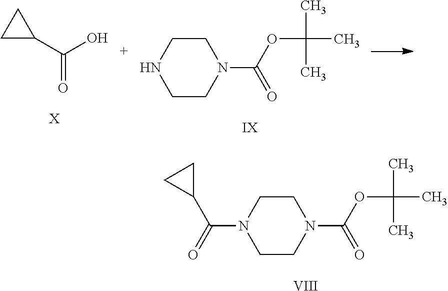

Cyclopropane carboxylic acid of formula X (50 g) was charged in RB flask, followed by dimethylacetamide (300 mL), to this carbonyl diimidazole i.e CDI (141 g) was added lot wise at 25-30° C., it was then stirred for 2 hours at 25-30° C. and then Boc-Piperazine of formula IX (162 g) was charged to it. Reaction mixture was stirred for 2 hours at 25-30° C. After completion of reaction, reaction mass was quenched in water and extracted the product with methylene dichloride (MDC) followed by aq. acetic acid washings and brine solution washing to organic layer. Organic layer was then concentrated and isolated the material from isopropyl ether (IPE) and dried the solid under vacuum at 45-50° C.

Yield: 105 g

### Example 48: Preparation of Compound of Formula VII

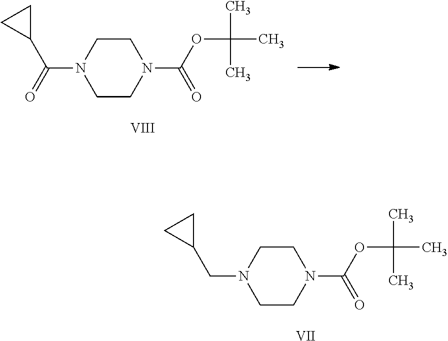

Compound of formula VIII (50 g) was changed to the RB flask along with tetrahydrofuran (100 mL) under Nitrogen atmosphere. Sodium borohydride (15 g) was added to it in lot wise at ambient temperature. Reaction mass was cooled to 0-10° C. BF₃-Etherate (85 mL) was added slowly to the reaction mass by maintaining temperature between 0-10° C. Reaction mass was then stirred for 2 hours at 0-10° C. After completion of reaction, reaction mass was quenched in ice water and extracted the product with methylene dichloride (MDC) followed by brine solution washings. The organic layer was then concentrated to get crude oil. Crude oil was stripped off with heptane and material was isolated from heptane as white solid.

Yield: 47 g

### Example 49: Preparation of Compound of Formula VIa

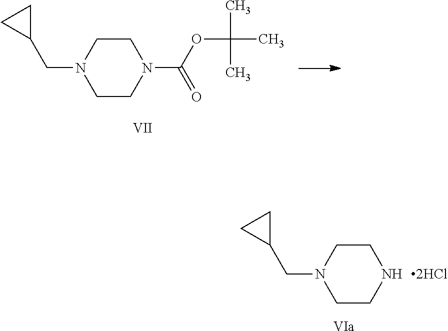

Compound of formula VII (40 g) and isopropyl alcohol (200 mL) was charged into RB flask. The temperature was raised to 45-50° C. and HCl (50 mL) was added to reaction mass at 45-50° C. Reaction mass was stirred for 3-4 hours at 45-50° C. After completion of the reaction, reaction mass was concentrated to residue and acetone (40 mL) was added and stripped off, acetone (400 mL) was added to it and stirred for 1 hour at 25-30° C., the solid was filtered off and washed with acetone (40 mL) followed by drying solid under vacuum at 45-50° C. to obtain white solid compound.

Yield: 35 g

### Example 50: Preparation of Compound of Formula IV

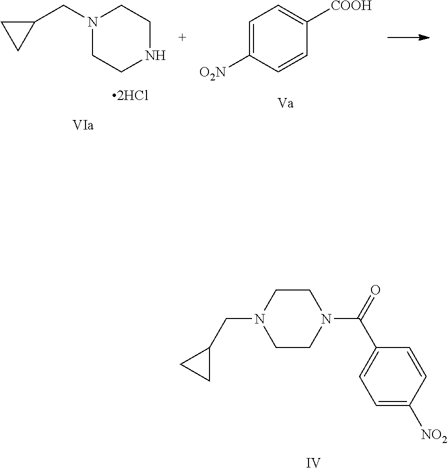

4-nitrobenzoic acid of formula Va (5 g), compound of formula VIa (4.2 g) and methylene dichloride (MDC) (50 mL) was charged into RB flask and cooled the reaction mass to 0-5° C. 1-(3-dimethylaminopropyl)-3-ethyl carbodiimide hydrochloride (EDAC. HCl) (7 g) was added followed by and 4-dimethylaminopyridine (DMAP) (0.75 g) at 0-5° C. Temperature of the reaction mass was raised to 25-30° C. and stirred the reaction mass for 2-3 hours at 25-30° C. After completion of reaction, reaction mass was quenched in water. Organic layer was washed with 5% citric acid solution followed by water and 20% sodium carbonate solution. Organic layer was then concentrated under vacuum at 40-45° C. to get residue which was taken for next stage.

Yield: 4.5 g

### Example 51: Preparation of Compound of Formula III

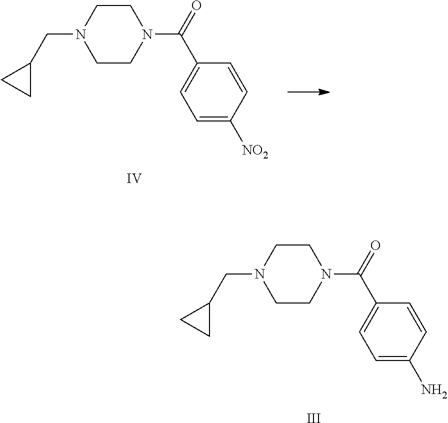 [SMILES: O=C(c1ccc([N+](=O)[O-])cc1)N1CCN(CC2CC2)CC1 ; "IV" ; Nc1ccc(C(=O)N2CCN(CC3CC3)CC2)cc1 ; "III"]

Compound of formula IV (4.5 g) was dissolved in methanol (45 ml), it was then hydrogenated with 5% Palladium on Carbon (0.5 g) using 3-5 kg hydrogen pressure at room temperature. Maintained the rection for 2-2.5 hours at room temperature under 3-5 kg hydrogen pressure. After completion of reaction, reaction mass was filtered off and filtrate was concentrated to get residue which was stripped off with isopropyl ether (IPE) followed by isolation from isopropyl ether (IPE) to get solid compound.

Yield: 3.5 g

### Example 52: Preparation of Mitapivat

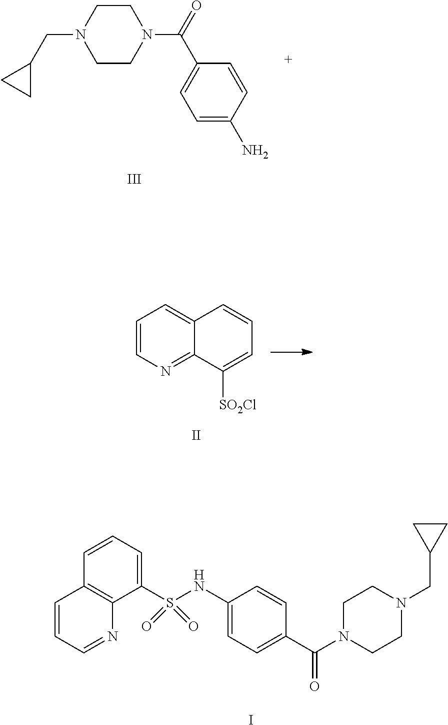

8-quinoline sulfonyl chloride of formula II (1.4 g), acetonitrile (10 ml) and (4-aminophenyl)-[4-(cyclopropylmethyl) piperazin-1-yl]methanone of formula III (1.5 g) was charged to the RB flask followed by acetonitrile (10 mL) and pyridine (0.7 mL) under nitrogen atmosphere. Reaction mass was stirred at 65-67° C. for about 4-6 hours. After completion of reaction water (80 mL) was charged to the reaction mass and extract the product with methylene dichloride (MDC). Washed the organic layer with water followed by distillation under vacuum at 40° C. and stripped off with isopropyl ether (IPE) followed by isolation of mitapivat from isopropyl ether (IPE).

Yield: 2.0 g

### Example 53: Preparation of Mitapivat

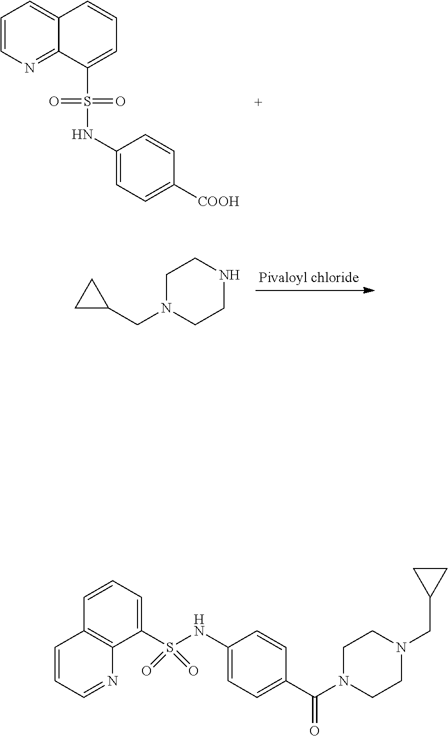 [SMILES: O=C(O)c1ccc(NS(=O)(=O)c2cccc3cccnc23)cc1 ; C1CN(CC2CC2)CCN1 ; O=C(c1ccc(NS(=O)(=O)c2cccc3cccnc23)cc1)N1CCN(CC2CC2)CC1]

Quinoline sulfonic acid (5 g), dichloromethane (50 mL) and add pivaloyl chloride (2.2 g) was charged into RB flask at 0-5° C., stirred for 10-15 minutes at 0-5° C. Added diisopropyl ethyl acetate (DIPEA) (2.5 g) very slowly at 0-10° C. and stirred for 2-2.5 hours at 0-10° C. Cyclopropane piperazine (2.7 g) in dichloromethane (5 mL) was added and stirred for 1 to 1.5 hours at 0-10° C., check TLC reaction quenched in water and organic layer washed with Aq. Sodium carbonate solution and washed with Aq. NaCl solution. The solvent was distilled completely and isolated in methanol.

### Example 54: Preparation of Mitapivat Mono Sulphate

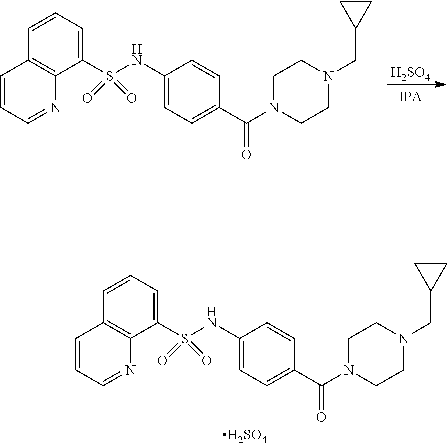

Isopropyl alcohol (12 mL) was charged in RB flask and cooled to 20-25° C. Sulfuric acid (1.95 g) was slowly added to it and cooled to 25-30° C. Mitapivat base (3 g) was added lot wise at 25-30° C. and the reaction mass was stirred for 15-16 hours at 25-30° C. Solid was filtered under N₂ atmosphere and washed the with Isopropyl alcohol (10 mL). Solid was dried under vacuum at 40-45° C. to get 3.5 gm of Mitapivat mono sulphate.

Yield: 3.5 g

1-13. (canceled)

14. A crystalline mitapivat hemi sulfate XI, characterized by Powder X-ray diffraction pattern having significant peaks at 2θ angles positions at about 12.55, 13.93, 16.82, 24.36 and 29.11±0.2°.

15. A process for preparation of crystalline mitapivat hemi sulfate Form XI according to claim 14, comprising drying crystalline mitapivat hemi sulfate Form IX or Form X or Form XII.

16. A crystalline mitapivat hemi sulfate X, characterized by Powder X-ray diffraction pattern having significant peaks at 2θ angles positions at about 7.21, 11.24, 12.19, 14.53 and 22.54±0.2°.

17. A process for preparation of crystalline mitapivat hemi sulfate Form X according to claim 16, comprising the steps of:
a) suspending mitapivat in a mixture of isobutyl acetate and trifluoro ethanol;
b) adding sulfuric acid diluted with isobutyl acetate to a reaction mixture obtained in step (a);
c) heating the reaction mixture to 40-50° C.;
d) cooling the reaction mixture to 20-30° C.; and
e) isolating the crystalline mitapivat hemi sulfate Form X.

18. A crystalline mitapivat hemi sulfate XII, characterized by Powder X-ray diffraction pattern having significant peaks at 2θ angles positions at about 6.58, 13.22, 15.18, 15.73, 19.82 and 20.75±0.2°.

19. A process for preparation of crystalline mitapivat hemi sulfate Form XII according to claim 18, comprising the steps of:
a) suspending mitapivat in a mixture of isobutyl acetate and trifluoro ethanol;
b) adding sulfuric acid diluted with isobutyl acetate to a reaction mixture obtained in step (a);
c) heating the reaction mixture to 40-50° C.;
d) cooling the reaction mixture to 20-30° C.;
e) optionally washing the reaction mixture obtained in step (d) with isobutyl acetate; and
f) isolating the crystalline mitapivat hemi sulfate Form XII.
